(19) United States
(12) Patent Application Publication
(10) Pub. No.: US 2003/0091620 A1
(43) Pub. Date: May 15, 2003

(54) Transdermal drug delivery systems containing quaternary ammonium salts and methods of using the same

(76) Inventors: David Fikstad, Salt Lake City, UT (US); Charles D. Ebert, Yorba Linda, CA (US); Srinivasan Venkateshwaran, So. Burlington, VT (US); Lawrence R. Nilssen, Bountiful, UT (US)

Correspondence Address: THORPE NORTH & WESTERN, LLP, M. Wayne Western, 8180 South 700 East, #200, Sandy, UT, 84070, US

(21) Appl. No.: 10/105,032

(22) Filed: Mar. 21, 2002

Related U.S. Application Data
(62) Division of application No. 09/657,080, filed on Sep. 7, 2000.
(60) Provisional application No. 60/153,001, filed on Sep. 8, 1999.
(60) Provisional application No. 60/153,008, filed on Sep. 8, 1999.
(60) Provisional application No. 60/153,015, filed on Sep. 8, 1999.

(51) Int. Cl. A61K 9/70

(52) U.S. Cl. 424/449

(57) ABSTRACT

A transdermal drug delivery system is disclosed, which includes a polymer, a drug and an amount of a quaternary ammonium salt that is sufficient to act as a penetration enhancer. The quaternary ammonium salt may also be present in an amount sufficient to act as an irritation reducer. Further, the transdermal drug delivery system may also contain a co-enhancer, which provides a synergistic skin permeation enhancing effect when combined with the quaternary ammonium salt. A method for enhancing the transdermal delivery of a drug is also disclosed.

### PRIORITY INFORMATION

This application claims priority to U.S. Provisional Patent Applications Serial No: 60/153,001, Serial. No: 60/153,008, and Serial. No: 60/153,015 each of which was filed on Sep. 9, 1999. Each of these applications is hereby incorporated by reference.

### FIELD OF THE INVENTION

The present invention relates generally to a transdermal drug delivery system containing a quaternary ammonium salt. Accordingly, this invention covers the fields of pharmaceutical sciences, medicine, and other health sciences.

### BACKGROUND OF THE INVENTION

Transdermal delivery of drugs provides many advantages over conventional oral administration. Such advantages include convenience, uninterrupted therapy, improved patient compliance, reversibility of treatment (by removal of the system from the skin), elimination of “hepatic first pass” effect, a high degree of control over blood concentration of the drug, and improved overall therapy.

Several compounds have been investigated as transdermal penetration enhancers to improve the flux of a drug across the skin. See, for example, U.S. Pat. Nos. 5,601,839; 5,006,342; 4,973,468, 4,820,720; 4,006,218, 3,551,154; and 3,472,931. Further, an index of permeation enhancers is disclosed by David W. Osborne and Jill J. Henke, in their internet publication entitled Skin Penetration Enhancers Cited in the Technical Literature, which may be found at the world wide web address pharmtech.comltechnicallosborne/osborne.htm, incorporated herein by reference in its entirety.

However, one challenge in the transdermal drug delivery has been to devise a formulation with improved penetration of drug molecules across the skin surface with reduced skin irritation. For example, Aoyagi, J. Controlled Release 13:63-71 (1990) describes a quaternary compound such as benzalkonium chloride at concentrations greater than 5% w/v as a penetration enhancer, but also notes that it causes severe irritation.

See also, U.S. Pat. Nos. 4,006,218, 4,505,901, and 5,346,886 for additional examples of quaternary ammonium salts as penetration enhancers. Accordingly, there is a need for novel transdermal formulations with good penetration characteristics and minimal irritation.

### SUMMARY OF THE INVENTION

The present invention provides a transdermal drug delivery system comprising a pharmaceutically acceptable carrier, a drug, and a quaternary ammonium salt as a penetration enhancer from about 0.1% to about 4.5% by weight of the carrier. In one aspect of the invention, the quaternary ammonium salt is a compound having the formula:

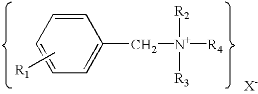

wherein R₁ is a member selected from the group consisting of H and C₁-C₁₂ straight or branched chain alkyl; R₂ and R₃ are independent members selected from the group consisting of CH₃, —CH₂OH and —CH₂ CH₂OH; R₄ is a member selected from the group consisting of:

(a) CH₃,

(b) C₂-C₂₂ straight or branched chain alkyl,

(c) C₂-C₂₂ straight or branched chain alkenyl,

(d) [CH₂CH₂O)]ₙ—R₅ where n is an integer of 1-3 and R₅ is a member selected from the group consisting of H, C₁-C₁₂ straight or branched chain alkyl, C₂-C₂₂ straight or branched alkenyl; and

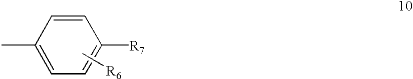

wherein R₆ is a member selected from the group consisting of H and —CH₃ and R₇ is a member selected from the group consisting of C₁-C₂₂ straight or branched chain alkyl and C₂-C₂₂ straight or branched chain alkenyl, and

(e)—(CH₂)ₘ NOCR₇ or —(CH₂)ₘ CONR₇ where m is an integer of 1-3 and R₇ is as described above; and

X is a pharmaceutically acceptable counter-ion or a mixture of counter ions.

Surprisingly, in addition to providing penetration enhancement, the quaternary ammonium salt may act as an anti-irritant at the concentrations disclosed herein. In one aspect of the invention, the quaternary ammonium salt is an alkyl-, dimethyl benzenemethanaminium salt; acyl-, dimethyl benzenemethanaminium salt; mixed acyl-/alkyl-, dimethyl benzenemethanaminium salt; ethylbenzyl dodecyl dimethylammonium chloride, dodecylbenzyltrimethylammonium chloride, dodecylbenzyl triethanolammonium chloride, benzoxonium chloride, benzethonium chloride; methylbenzethonium chloride; phenoctide; dodecarbonium chloride; and mixed alkyl-/acyl-, amidopropalkonium salt, or a mixture thereof.

While the pharmaceutically acceptable carrier may comprise any acceptable material, in one aspect, it comprises a biocompatible polymer. In another aspect, the carrier may be an adhesive. In another aspect, the pharmaceutically acceptable carrier comprises a viscous material, which is suitable for inclusion in a liquid reservoir.

In one aspect of the invention, the adhesive may be, but is not limited to, one or more of the following: acrylics, vinyl acetates, natural and synthetic rubbers, ethylene-vinyl acetate copolymers, polysiloxanes, polyacrylates, polyurethanes, plasticized polyether block amide copolymers, plasticized styrene-rubber block copolymers, and mixtures thereof. In another aspect of the invention, the viscous material may form a gel.

The transdermal drug delivery system of the present invention may also include one or more additives known in the art, such as diluents, excipients, emollients, plasticizers, skin irritation reducing agents, carriers and co-enhancers as described herein. In some aspects, the co-enhancer acts synergistically with the quaternary ammonium salt to enhance the penetration of the drug.

In some aspects, the co-enhancer is a compound represented by the formula:

R-Y

wherein R is a straight chain alkyl of about 7 to 17 carbon atoms, a non-terminal alkenyl of about 7 to 22 carbon atoms, or a branched-chain alkyl from about 12 to 22 carbons; and Y is —OH, —COOH, —OCOCH₃, —SOCH₃, —P(CH₃)₂O, —COO(C₂H₄O)ₘH, —(OC₂H₄)ₘOH, —COOCH₂CH(OH)CH₃, —COOCH₂CH(OH)CH₂OH, —COOCH₂CHXCH₂X, —CO(OCH₂CO)ₙOM, —CO[OCH(CH₃)CO]ₙOM —COOCH[CH(OH)]₄CH₂OH, —CO[C₆H₁₂O₆, sucrose], —CONR¹R², —COO(CH₂)₂NR¹R², —COO[CH(CH₃)CH₃]NR¹R², —COOR³, or N-pyrrolidone; where X is H or RCOO—; M is H or a pharmaceutically acceptable counter ion; R¹ and R² are independently H, CH₃, C₂H₅, C₃H₇, C₂H₄OH, or C₃H₇OH; R³ is CH₃, C₂H₅, or C₃H₇; m is an integer of 2 to 6; and n is an integer of 1 to 4. In some aspects, the co-enhancer is glycerol, or a glyceryl compound such as glyceryl monooleate, glyceryl dioleate, glyceryl trioleate, etc. In another aspect, the co-enhancer is triacetin.

The counter-ion of the present invention can be any pharmaceutically acceptable counter-ion. Several such counter-ions are well known in the art. Some examples include, but are not limited to: chloride, bromide, iodide, acetate, 2-ethylhexanoate, sulfate, phosphate, arylsulfonates, cyclohexylsulfamate, benzoate, saccharinate, and a mixture thereof.

A broad range of drugs may be delivered using the transdermal drug delivery system of the present invention. Several examples are presented below. Practically any drug belonging to any therapeutic class may be delivered.

Methods are also provided for enhancing transdermal delivery of a drug and reducing skin irritation associated with such transdermal delivery. In one aspect, such a method includes the step of applying a transdermal delivery system, as disclosed herein, to a selected skin surface.

### DETAILED DESCRIPTION

### A. DEFINITIONS

In describing and claiming the present invention, the following terminology will be used.

The singular forms “a,” “an,” and “the” include plural referents unless the context clearly dictates otherwise. Thus, for example, reference to “a drug” includes reference to one or more drugs, and reference to “an enhancer” includes reference to one or more of such enhancers.

A “quaternary ammonium salt” refers to a tetravalent nitrogen-containing molecule with a positive charge on nitrogen and a counter ion. Such quaternary ammonium salts include aliphatic and aromatic substituents. One example of an aliphatic quaternary ammonium salt is a tetraalkyl ammonium chloride, such as tetramethyl ammonium chloride, tetraethyl ammonium chloride, etc. An example of an aromatic quaternary ammonium salt is a quaternary benzyl ammonium salt (“benzyl quaternary ammonium salts,” “benzyl quaternary ammonium compound”) and refer to a compound with the formula:

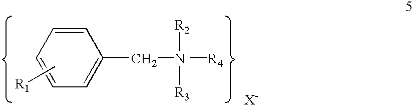

wherein R₁ is a member selected from the group consisting of H and C₁-C₁₂ straight or branched chain alkyl; R₂ and R₃ are independent members selected from the group consisting of CH₃, —CH₂OH and —CH₂ CH₂OH; R₄ is a member selected from the group consisting of:

(a) CH₃,

(b) C₂-C₂₂ straight or branched chain alkyl,

(c) C₂-C₂₂ straight or branched chain alkenyl,

(d) [CH₂CH₂O)]ₙ—R₅ where n is an integer of 1-3 and R₅ is a member selected from the group consisting of H, C₁-C₁₂ straight or branched chain alkyl, C₂-C₂₂ straight or branched alkenyl; and

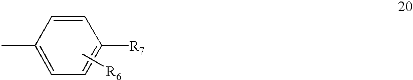

wherein R₆ is a member selected from the group consisting of H and —CH₃ and R₇ is a member selected from the group consisting of C₁-C₂₂ straight or branched chain alkyl and C₂-C₂₂ straight or branched chain alkenyl, and

(e)—(CH₂)ₘ NOCR₇ or —(CH₂)ₘ CONR₇ where m is an integer of 1-3 and R₇ is as described above; and

X is a pharmaceutically acceptable counter ion. Such counter ions are well known in the art. Some examples include chloride, bromide, iodide, acetate, 2-ethylhexanoate, sulfate, phosphate, arylsulfonates, cyclohexylsulfamate, benzoate, saccharinate, and a mixture thereof.

The terms “formulation” and “composition” are used interchangeably herein.

The terms “permeant,” “bioactive agent,” “pharmaceutical,” and “drug” are also used interchangeably and refer to a pharmacologically active substance or composition. These terms of art are well known in the pharmaceutical and medicinal arts.

As used herein, “transdermal” or “percutaneous” delivery refers to delivery of a drug by passage into and through the skin or mucosal tissue for systemic delivery or for localized treatment without systemic uptake. Transdermal administration can be accomplished by applying, pasting, rolling, attaching, pouring, pressing, rubbing, etc., of a transdermal preparation onto a skin surface. These and additional methods of administration are well known in the art.

“Transdermal drug delivery system” refers to a composition comprising a polymer and a drug for transdermal delivery across a skin surface. Additional ingredients may be added, including penetration enhancers, diluents, skin irritation reducing agents, excipients, plasticizers, emollients, or mixtures thereof. Examples of specific embodiments of a transdermal drug delivery system include but are not limited to non-patch topical formulations (such as ointments, creams, gels, lotions, sprays, foams, and pastes) and transdermal patch devices such as matrix patch devices and liquid reservoir patch devices.

One example of a transdermal patch in accordance with the present invention is a matrix-type patch which comprises a backing that is impermeable to a drug and defines the face or top surface of the patch and a solid or semisolid matrix layer comprising the drug, a biocompatible polymer, a quaternary ammonium salt permeation enhancer, and optionally a co-enhancer. In some aspects, the polymer is a pressure sensitive adhesive. In some aspects, the backing is occlusive, whereas in other aspects, the backing is non-occlusive (i.e., breathable). Matrix patches are known in the art of transdermal drug delivery. See, for example, U.S. Pat. Nos. 5,122,383 and 5,460,820, which are incorporated by reference in their entirety.

Another example of a transdermal patch for administering a drug in accordance with this invention is a liquid reservoir system (LRS) type patch, which comprises a drug, a quaternary ammonium salt permeation enhancer, and optionally a co-enhancer, in a carrier vehicle. The carrier vehicle comprises a fluid of desired viscosity, such as a gel or ointment, which is formulated for confinement in a reservoir having an impermeable backing and a skin contacting permeable membrane, or membrane adhesive laminate providing diffusional contact between the reservoir contents and the skin. For application, a peelable release liner is removed and the patch is attached to the skin surface. LRS patches are known in the art of transdermal drug delivery. Examples without limitation, of LRS transdermal patches are those described or referred to in U.S. Pat. Nos. 4,849,224, 4,983,395, which are incorporated by reference in their entirety. “Pharmaceutically acceptable carrier” refers to any pharmaceutically acceptable material that makes up a substantial part of the formulation. The carrier may be polymeric or non-polymeric and is admixed with other components of the composition (e.g., drug, binders, fillers, penetration enhancers, anti-irritants, coloring agents, sweeteners, flavoring agents, etc, as needed) to comprise the formulation.

The term “admixed” means that the drug and/or enhancer can be dissolved, dispersed, or suspended in the carrier.

“Skin,” “skin surface,” “derma,” and “epidermis,” are used interchangeably herein, and refer to not only the outer skin of a subject comprising the epidermis, but also to mucosal surfaces to which a drug composition may be administered. Examples of mucosal surfaces include the mucosa of the respiratory (including nasal and pulmonary), oral (mouth and buccal), vaginal, labial, and rectal surfaces. Hence the term “transdermal” encompasses “transmucosal.”

“Enhancement,” or “permeation enhancement,” may be used interchangeably, and refer to an increase in the permeability of the skin, to a drug, so as to increase the rate at which the drug permeates through the skin. Thus, “permeation enhancer” or “penetration enhancer” or simply “enhancer” refers to an agent, or mixture of agents that achieves such permeation enhancement. In one aspect, the increase in permeation is measured by comparing to a formulation that has no enhancer or an enhancer that is of a different kind or in different concentration. Other general methods for measuring penetration enhancement are well known in the art. For example, the methods described in Merritt et al., Diffusion Apparatus for Skin Penetration, J. of Controlled Release 61 (1984), incorporated herein by reference in its entirety. Further methods include those disclosed in U.S. Pat. Nos. 4,863970, 4,888,354, 5,164,190, and 5,834,010, which are incorporated by reference in their entirety.

An “effective amount” of an enhancer means an amount effective to increase penetration of a drug through the skin, to a selected degree. Methods for assaying the effective amount and other characteristics of permeation enhancers are well known in the art. See, for example Merritt et al. at 61.

“Therapeutically effective amount,” refers to a sufficient amount of a drug, to achieve therapeutic results in treating a condition for which the drug is expected to be effective. The determination of an effective amount is well within the ordinary skill in the art of pharmaceutical and medical sciences. See for example, Curtis L. Meinert & Susan Tonascia, Clinical Trials: Design, Conduct, and Analysis, Monographs in Epidemiology and Biostatistics, vol. 8 (1986).

A “low concentration, ” and “low amount,” as used with reference to a quaternary ammonium salt means a concentration of a quaternary ammonium salt, which is about 4.5%, or less by weight of a pharmaceutical carrier into which the quaternary ammonium salt is incorporated.

Concentrations, amounts, solubilities, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited.

For example, a concentration range of about 1% w/w to about 4.5% w/w should be interpreted to include not only the explicitly recited concentration limits of 1% w/w to about 4.5% w/w, but also to include individual concentrations such as 2% w/w, 3% w/w, 4% w/w, and sub-ranges such as 1% w/w to 3% w/w, 2% w/w to 4%w/w, etc. The same principle applies to ranges reciting only one numerical value, such as “less than about 4.5% w/w,” which should be interpreted to include all of the above-recited values and ranges. Further, such an interpretation should apply regardless of the breadth of the range or the characteristic being described.

“Reduced irritation” refers to a reduction in skin irritation as evidenced by a decrease in the incidence or severity of inflammation, lesions, erythema, lichenification, blistering, edema, desquamation, fissuring, necrosis, escharing, blanching, etc. A reduction in irritation may be measured by both visual observations, for example, using a Visual Analog Scale, and patient comfort indication. General methods of evaluating primary skin irritation, including reductions of irritation are disclosed in the protocol of Springborn Laborotores entitled A primary Skin Irritation Study in Rabbits, Springborn Laboratories (1998); see also, SOT Position Paper, Comments on the LD50 and Acute Eye and Skin Irritation Tests, Fundamental and Applied Toxicology 13:621-623, (1989), which are incorporated herein in their entirety.

### B. THE INVENTION

The present invention provides a transdermal drug delivery system comprising a quaternary ammonium salt as a penetration enhancer. In addition to enhancing the penetration of various drugs, the quaternary ammonium salt may also act as an anti-irritant, to reduce skin irritation induced by the application of a transdermal drug delivery system to the skin. Further, a second penetration enhancer (“co-enhancer”) may be combined with the quaternary ammonium salt for synergistic penetration enhancing effect.

### a) General Aspects

The transdermal drug delivery system may take a variety of well-known delivery formulations, including but not limited to adhesive matrix patches, liquid reservoir system (LRS) patches, transmucosal patches or tablets, and topical formulations, such as creams, lotions, ointments, etc. Examples of such pharmaceutical formulations may be found in the references listed in the definitions section above.

In one general aspect, the transdermal drug delivery system comprises a pharmaceutically acceptable carrier, a drug for transdermal delivery, and a quaternary ammonium salt comprising about no greater than 4.5% by weight of the carrier.

When presented in the form of a transdermal patch, the transdermal drug delivery system of the present invention may include structural components, as known in the art. For example, in the case of an adhesive matrix patch, a distal backing is laminated to the polymer layer. Such a distal backing defines the side of the matrix patch that faces the environment, i.e., distal to the skin or mucosa. The backing layer functions to protect the matrix polymer layer and drug/enhancer composition and to provide an impenetrable layer that prevents loss of drug to the environment. Thus, the material chosen for the backing should be compatible with the polymer layer, drug, and enhancer, and should be minimally permeable to any components of the matrix patch. Advantageously, the backing can be opaque to protect components of the matrix patch from degradation from exposure to ultraviolet light. Furthermore, the backing should be capable of binding to and supporting the polymer layer, yet should be pliable enough to accommodate the movements of a person using the matrix patch.

Suitable materials for the backing include, but are not limited to: metal foils, metalized polyfoils, composite foils or films containing polyester such as polyester terephthalate, polyester or aluminized polyester, polytetrafluoroethylene, polyether block amide copolymers, polyethylene methyl methacrylate block copolymers, polyurethanes, polyvinylidene chloride, nylon, silicone elastomers, rubber-based polyisobutylene, styrene, styrene-butadiene and styrene-isoprene copolymers, polyethylene, and polypropylene. In one aspect of the invention, the backing layer may have a thickness of about 0.0005 to 0.01 inch.

Further, a release liner may be temporarily provided upon the proximal side (side to adhere to the skin) of the adhesive layer. Such a liner provides many of the same functions as the backing layer, prior to adhesion of the patch to the skin. In use, the release liner is peeled from the adhesive layer just prior to application and discarded. The release liner can be made of the same materials as the backing layer, or other suitable films coated with an appropriate release surface.

### b) The Carrier

The pharmaceutically acceptable carrier of the present transdermal drug delivery device may be made of a wide variety of materials known to those skilled in the art of transdermal drug delivery. In one aspect of the invention the carrier is a biocompatible polymer. In another aspect, the carrier is an adhesive. In the case of an adhesive matrix patch, the carrier is a biocompatible adhesive polymer. The carrier, in some aspects, may contain both the drug to be transdermally delivered, and a quaternary ammonium salt. In the case of an LRS patch, the carrier forms a gel, or other viscous form suitable for use in an LRS patch as is known in the art. Such a viscous carrier may contain both the drug to be transdermally delivered as well as a quaternary ammonium salt. Further, a quaternary ammonium salt may be incorporated into the adhesive portion of an LRS patch, which does not contain any drug, but is used primarily to hold the reservoir against the skin.

In one aspect, the pressure-sensitive adhesive of the pharmaceutically acceptable carrier is suitable for long-term (e.g., greater than 1 day, may be about 3-4 days, or longer such as 1-4 weeks) contact with the skin. In another aspect, the pressure-sensitive adhesive of the carrier is suitable for a short-term administration (e.g., for a few minutes to a few hours, less than or equal to 1 day). Such adhesives must be physically and chemically compatible with the drug and enhancer, and with any carriers and/or vehicles or other additives incorporated into the drug/enhancer composition. In one aspect of the invention, the adhesives of the pharmaceutically acceptable carrier include without limitation, acrylic adhesives including cross-linked and uncross-linked acrylic copolymers; vinyl acetate adhesives; natural and synthetic rubbers including polyisobutylenes, neoprenes, polybutadienes, and polyisoprenes; ethylenevinylacetate copolymers; polysiloxanes; polyacrylates; polyurethanes; plasticized weight polyether block amide copolymers, and plasticized styrene-rubber block copolymers or mixtures thereof. In yet another aspect of the invention, contact adhesives for use in the pharmaceutically acceptable carrier layer are acrylic adhesives, such as DuroTak® 87-2888 adhesive (National Starch & Chemical Co., Bridgewater, N.J.); and polyisobutylene adhesives such as ARcare™ MA-24 (Adhesives Research, Glen Rock, Pa.) and ethylene vinyl acetate copolymer adhesives.

While the pharmaceutically acceptable carrier of an LRS patch may be of any suitable viscous material known to those skilled in the art of transdermal drug delivery, in one aspect of the present invention, the pharmaceutically acceptable carrier of the liquid reservoir forms a gel.

In addition to containing the drug and quaternary ammonium salt, the pharmaceutically acceptable carrier may comprise a number of other additives, such as diluents, excipients, emollients, plasticizers, skin irritation reducing agents, or a mixture thereof. These types of components, as well as others not specifically recited, are well known in the art for inclusion in various transdermal formulations, and may be added as desired to the transdermal drug delivery system of the present invention in specific types and amounts in order to achieve a desired result.

For example, suitable diluents can include mineral oil, low molecular weight polymers, plasticizers, and the like. Many transdermal drug delivery formulations have a tendency to cause skin irritation after prolonged exposure to the skin, thus addition of a skin irritation reducing agent aids in achieving a composition that is better tolerated by the skin. In one aspect, the skin irritation reducing agent may be glycerin, as disclosed in U.S. Pat. No. 4,855,294, which is incorporated by reference in its entirety.

### c) The Drug

As described above, the present invention can be used to deliver a wide variety of drugs, including vitamins, diagnostic agents, cosmetic agents, macromolecules, etc. One of ordinary skill in the art would appreciate that practically any drug or other desired transdermally effective agent is a suitable candidate for delivery.

In general, drugs for use in the present composition include therapeutic agents in all of the therapeutic areas including, but not limited to: antibiotics (including antimicrobials, antibacterials, antimycobacterials, antimalerials, antiamebics, anthelminics, antifungals, and antivirals), neoplastic agents, agents affecting the immune response (including steroidal and non-steroidal anti-inflammatory agents), blood calcium regulators, peptide and protein hormones, agents useful in glucose regulation, antithrombotics and hemostatics, antihyperlipidemic agents, thyromimetic and antithyroid drugs, antiulcer agents, histamine receptor agonists and antagonists, inhibitors of allergic response, local anesthetics, analgesics and analgesic combinations, antipsychotics, anti-anxiety agents, antidepressants agents, anorexigenics, bone-active agents, diagnostic agents, and a mixture thereof. Additional examples include: antidiarrheals, antimigraine preparations, antimotion sickness agents, antinauseants, antiparkinsonism drugs, antipruritics, antipyretics, antispasmodics (including gastrointestinal, urinary, skeletal, and smooth-muscle), anticholinergics, sympathomimetics, xanthine derivatives, cardiovascular preparations (including calcium channel blockers, beta-blockers, antiarrythmics, antihypertensives, diuretics, vasodilators including general coronary, peripheral and cerebral), central nervous system stimulants including cough and cold preparations, decongestants, diagnostics, hormones, immunosuppressives, parasympatholytics, parasympathomimetics, sedatives, tranquilizers and mixtures thereof.

Examples of specific drugs include without limitation: antibiotics: amoxicillin, cloxacillin sodium, penicillin G potassium; antimicrobials: benzalkonium chloride, chlorohexidine, gluconate hexachlorophene; antibacterials: sulfabenzamide, sulfadiazine, sulfasalazine; antimycobacterials: chlofazimine, ethambutol, isoniazid; antimalerials: chloroquine hydrochloride, quinine sulfate, pyrimethamine; antiamebics: arsthinol, bialamicol, carbarsone; anthelminics: ivermectin, bithionol, piperazine; antifungals: clotrimazole, griseofulvin, miconazole; antivirals: acyclovir, foscarnet sodium, ribavirin; neoplastic agents: adriamycine, cyclophosphamide, methotrexate; immune response steroidal anti-inflammatory agents: hydrocortisone, dioxyanthranol, betamethasone; non-steroidal anti-inflammatory agents (NSAIDs): choline salicylate, diflunisal, ibuprofen, acetaminophen; blood calcium regulators: parathyroid hormone, calcifediol, calcitonin; peptide and protein hormones: insulin, glucagon, vasopressin; glucose regulators: tolazamide, tolbutamide, chlorpropamide; antithrombotics: aspirin, sulfinpyrazone, dipyridamole; hemostatics: thrombin, microfibrillar collagen, absorbable gelatin powder; antihyperlipidemic agents: pravastatin sodium, simvastatin, clinofibrate; thyromimetic and antithyroid drugs: methimazole, propylthiouracil, potassium iodide; antiulcer agents: metaclopramide, histidine hydrochloride, famotidine; histamine receptor agonists and antagonists: astemizole, clemastine fumarate, cyclizine; allergic response inhibitors: astemizole, clemastine fumerate, diphenhydramine hydrochloride; local anesthetics: chloroprocaine hydrochloride, lidocaine hydrochloride, procaine hydrochloride; analgesics and analgesic combinations: acetaminophen, aspirin, ibuprofen; antipsychotics: acetophenazine maleate, chlorprothixene, droperidol; anti-anxiety agents: diphenhydramine, phenobarbital, chlordiazepoxide; anti-depressants: amitriptyline hydrochloride, amoxapine, fluoxetine hydrochloride; anorexigenics: amphetamine, methamphetamine, chlorphentermine; bone-active agents: parathyroid hormone, calcitonin; diagnostic agents: benzylpeniclloyl polylysine, iocetamic acid, aminohippurate sodium; antidiarrheals: diphenoxylate hydrochloride, loperamide hydrochloride, fennel oil; antimigraine preparations: dihydroergotamine mesylate, ergotamine tartrate, methysergide maleate, sumatriptin succinate; antimotion sickness agents: buclizine hydrochloride, diphenidol, meclizine hydrochloride; antinauseants: benzquinamide hydrochloride, dronabinol, dimenhydrinate; antiparkinsonism drugs: amantadine hydrochloride, benztropine mesylate, biperiden hydrochloride; antipruitics: camphor, menthol, pramoxine; antipyretics: acetaminophen, aspirin, ibuprofen; antispasmodics (including gastrointestinal, urinary, skeletal and smooth-muscle): flavoxate, flavoxate hydrochloride, ethaverine hydrochloride, oxybutynin chloride, dicyclomine; anti-cholinergics: propantheline, oxybutynin, oxybutynin hydrochloride, adiphenine hydrochloride, aminopentamide, atropine; sympathomimetics: dopamine hydrochloride, epinephrine, ephedrine sulfate; xanthine derivatives: caffeine, theophylline, aminophylline; calcium channel blockers: amlodipine, felodipine, isradipine, diltiazem, nifedipine; beta blockers: propanolol, pindolol, labetalol, betaxolol; anti-arrythmics: procainamide, prajmaline, disopyramide; antihypertensives: clonidine hydrochloride, guanabenz acetate, methyidopa; diuretics: ammonium chloride, mannitol, urea, hydrochlorothiazide, bumetanide; vasodilators: (general) diazoxide, minoxidil, pinacidil; (Coronary) amotriphene, bendazol, benfurodil hemisuccinate; (Peripheral) bamethan, bencyclane, betahistine; (Cerebral) bencyclane, cinnarizine, citicoline; central nervous system (CNS) stimulants cough and cold preparations: dextromethorphan hydrobromide; decongestants: pseudoephedrine hydrochloride, diphenhydramine hydrochloride; chlorpheniramine maleate; hormones: estradiol, corticosteroids, hydrocortisone; testosterone, progesterone; immunosuppressives: cyclosporin, mizoribine, brequinar sodium; parasympatholytics: atropine sulfate, belladonna, cyclopentolate hydrochloride; parasympathomimetics: pyridostigmine, physostigmine, scopolamine; sedatives: buspirone hydrochloride, chloral hydrate, disulfiram; tranquilizers: chloropromazine, promazine, fluphenzaine.

In some aspects, the drug may be oxybutynin, buspirone, fentanyl, testosterone, progestin, estradiol, propentofylline, or a mixture thereof. It should be appreciated that one or more of these and other drugs described herein exist in many pharmaceutically acceptable salts. Examples of such salts include those generated by using inorganic agents (i.e., inorganic cations such as sodium, potassium, calcium, etc., and inorganic anions such as chloride, bromide, etc.,) and organic agents (i.e., organic cations such as piperazinyl, triazinyl, etc., and organic anions such as citrates, tartarates, tosylates, etc). In addition, these drugs are also present as polymorphs and/or isomers. Examples of polymorphs include monohydrates, dihydrates, hemi-hydrates, etc., as well those high-melting and low-melting polymorphs. These polymorphs can be characterized using X-ray crystallographic techniques or other well-known techniques in the art. Examples of isomers include geometric and optical isomers. Further, the pharmaceutical art has recognized that such salts, isomers, and polymorphs, as well as prodrugs, analogs, and metabolites for these drugs can be therapeutically effective as well and can be substituted with ease.

Examples of useful testosterone and related compounds include without limitation: testosterone, methyltestosterone, androstenedione, adrenosterone, dehydroepiandrosterone, oxymetholone, fluoxymesterone, methandrostenolone, testosterone, methyltestosterone, androstenedione, adrenosterone, dehydroepiandrosterone, oxymetholone, fluoxymesterone, methandrostenolone, testolactone, pregnenolone, 17α-methylnortestosterone, norethandrolone, dihydrotestosterone, danazol, oxymetholone, androsterone, nandrolone, stanozolol, ethylestrenol, oxandrolone, bolasterone and mesterolone, testosterone propionate, testosterone cypionate, testosterone phenylacetate, testosterone enanthate, testosterone acetate, testosterone buciclate, testosterone heptanoate, testosterone decanoate, testosterone caprate, testosterone isocaprate, and combinations thereof.

These testosterone compounds can be present in subsaturated concentrations, or low concentrations. Examples of compositions comprising subsaturated testosterone are known in the art. See, for example, U.S. Pat. Nos. 5,164,190, and 5,152,997, which are incorporated herein by reference. These testosterone compositions and/or other sex hormones, such as estrogen, progestin, etc. can also be provided using carriers that are stable over long-term storage. Such compositions may comprise ethylhexylacrylate polymers, as descrived in U.S. Pat. No. 5,780,050, which is incorporated by reference herein. Methods for providing such hormones to males and females are also well known. See, U.S. Pat. Nos. 5,460,820, 5,152,997, and 5,783,208, which are incorporated by reference herein. It is appreciated that using the disclosure of the present invention, one skilled in the art can readily accomplish the objective of the above-referenced patents.

Examples of useful estradiol and related compounds include without limitation: 17β-estradiol, 17α-estradiol, conjugated equine estrogen, esterified estrogen, micronized estradiol, sodium estrogen sulfate, ethinyl estradiol, estrone, tibolone, selective estrogen receptor modulator (SERM), phytoestrogen, and mixtures thereof. Examples of useful progestin and related compounds include without limitation: progesterone, medroxy-progesterone acetate, norethindrone, and norethindrone acetate.

Examples of useful oxybutynin compounds include without limitation: N-desethyloxybutynin, (R)-oxybutynin, (S)-oxybutynin, (R)-N-desethyloxybutynin, and (S)-N-desethyloxybutynin. Particularly, it has been noted that the oxybutynin metabolite, N-desethyloxybutynin, as well as it (R)- and (S)- optical isomers exert an anticholinergic action that is equal to or greater than oxybutynin, and can be readily delivered for such a purpose. See, U.S. Pat. Nos. 5,411,740, 5,500,222, 5,532,278, 5,677,346, 5,686,097, 5,736,577, 5,747,065, 5,750,137, and 5,900,250, which are incorporated by reference in their entirety.

Transdermal delivery of oxybutynin using triacetin as a penetration enhancer has been described by U.S. Pat. Nos. 5,834,010, and 5,601,839, which are incorporated herein by reference. It is appreciated that transdermal penetration of oxybutynin can be enhanced further by using a quaternary ammonium salt as described by the present invention, and triacetin as a co-enhancer. Oxybutynin can be administered in low concentrations, such that the serum concentrations of one or more of its metabolites can be significantly lowered with the beneficial effect of reduced adverse drug reactions, such as anticholinergic effects (including dry mouth, constipation, blurred vision, etc.). For example, such compositions may comprise an amount of oxybutynin, such that when administered to a subject a plasma area under the curve (AUC) ratio of oxybutynin to an oxybutynin metabolite is from about 0.5:1 to about 5:1. Such oxybutynin compositions have been described in co-pending application Ser. No. 09/559,711 filed on Apr. 26, 2000, which is incorporated herein by reference.

Examples of propentofylline compositions, which can be used in connection with the present invention are described in U.S. Pat. No. 5,762,953, which is incorporated herein by reference. It is appreciated that the transdermal penetration of such compositions may be further enhanced using the quaternary ammonium salt compounds of the present invention.

It is appreciated that any combination of any of the above drugs (that is one or more of any of the above drugs) may be used in this invention. The present invention also contemplates the use of such salts, isomers, polymorphs, prodrugs, analogs, and metabolites, including substances not specifically recited above.

It should also be recognized that the term “drug” as used herein refers to practically any chemical substance that has pharmacological activity or biological activity, as well as those substances that can be used for diagnostic or cosmetic purposes. Thus, vitamins, such as vitamin A, C, E, K, and various B complexes, veterinary drugs, and cosmetic agents such as wrinkle-reducing agents (including anti-oxidants, for example, ascorbic acid, ascorbyl palmitate, catechins, an polyphenol compounds), depilating agents (including calcium salt, thioglycolic acid, and calcium hydroxide), hair-growing agents (including relaxin, cyproterone acetate, spironolactor, flutamide, and minoxidil), depigmenting agents (including sulfites, bisulfites, and metabisulfites, and alkaline earth, and alkaline earth metal compounds thereof), are also included. Further, the term “drug” includes peptides, proteins, carbohydrates, fats, etc that are known to exert biological and or pharmacological effects.

It is appreciated that the above categories of drugs are not rigidly described and that one drug may be described accurately in more than one category or subcategory. For example, insulin may be described as a hormone, as an anti-diabetic agent and also as a macromolecule.

### d) The Quaternary Ammonium Salt

The quaternary ammonium salt that is suitable for this invention may be an aliphatic or aromatic compound. Examples of aliphatic quaternary ammonium salts include, but not limited to, alkyl quaternary ammonium salts such as tetramethyl ammonium chloride, tetraethyl ammonium chloride, etc. Examples of aromatic quaternary ammonium salts include benzalkonium chloride, benzethonium chloride, etc. In one aspect, the quaternary ammonium salt is a compound having the formula:

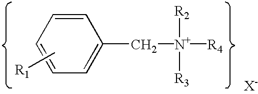

wherein R₁ is a member selected from the group consisting of H and C₁-C₁₂ straight or branched chain alkyl; R₂ and R₃ are independent members selected from the group consisting of CH₃, —CH₂OH and —CH₂CH₂OH; R₄ is a member selected from the group consisting of:

(a) CH₃,

(b) C₂-C₂₂ straight or branched chain alkyl,

(c) C₂-C₂₂ straight or branched chain alkenyl,

(d) [CH₂CH₂O—]ₙ—R₅ where n is an integer of 1-3 and R₅ is a member selected from the group consisting of H, C₁-C₁₂ straight or branched chain alkyl, C₂- C₂₂ straight or branched alkenyl; and

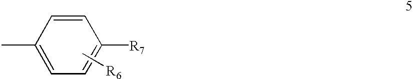

wherein R₆ is a member selected from the group consisting of H and —CH₃ and R₇ is a member selected from the group consisting of C₁-C₂₂ straight or branched chain alkyl and C₂-C₂₂ straight or branched chain alkenyl, and

(e)—(CH₂)ₘ NOCR₇ or —(CH₂)ₘ CONR₇ where m is an integer of 1-3 and R₇ is as described above; and

X is a pharmaceutically acceptable counter-ion.

In another aspect of the invention, the quaternary ammonium salt may be benzalkonium chloride; benzalkonium saccharinate; behenalkonium chloride; cetalkonium chloride; erucalkonium chloride; lauralkonium chloride; myristalkonium chloride; myristalkonium saccharinate (Quaternium-3); stearalkonium chloride; olealkonium chloride; tallowalkonium chloride; dodecylbenzyltrimethylammonium chloride (Quaternium-28); dodecylbenzyl trimethyl ammonium 2-ethylhexanoate; ethylbenzyl alkyldimethylammonium cyclohexylsulfanamate (Quaternium-8); ethylbenzyl dimethyl dodecyl ammonium chloride (Quaternium-14); dodecylbenzyl dimethyl octadecyl amrnmonium chloride; dodecylbenzyl triethanol ammonium chloride (Quaternium-30); benzoxonium chloride; benzylbis(2-hydroxyethyl)(2-dodecyloxyethyl)ammonium bromide; benzylbis(2-hydroxyethyl)(2-dodecyloxyethyl)ammonium chloride; benzethonium chloride; methylbenzethonium chloride;N,N-(diethyl-N-[2-[4-( 1,1,3,3 -tetramethylbutyl)phenoxy]ethyl] benzenemethanaminium chloride (phenoctide); dodecarbonium chloride; babassuamidopropalkonium chloride; and wheatgermamidopropalkonium chloride.

In another aspect of the invention, the quaternary ammonium is benzalkonium chloride, stearalkonium, behenalkonium chloride, olealkonium chloride, erucalkonium chloride, benzethonium chloride, methylbenzethonium chloride, phenoctide, wheatgermamidopropalkonium chloride and babassuamidopropalkonium chloride, or a mixture thereof. In another aspect of the invention, the quaternary ammonium salt enhancer is benzethonium chloride. In a further aspect of the invention, the quaternary ammonium salt is methylbenzethonium chloride. In another aspect of the invention, the quaternary an mnonium salt is benzalkonium chloride. In yet another aspect of the invention, the quaternary ammonium salt is olealkonium chloride. In another aspect of the invention the quaternary ammonium salt is phenoctide.

In one aspect of the invention, the quaternary ammonium salt is a member selected from the group consisting of alkyl-, dimethyl benzenemethanaminium salts; acyl-, dimethyl benzenemethanaminium salts; mixed acyl-/alkyl-, dimethyl benzenemethanaminium salts; ethylbenzyl dodecyl dimethylammonium chloride, dodecylbenzyltrimethylammonium chloride, dodecylbenzyl triethanolammonium chloride, benzoxonium chloride, benzethonium chloride; methylbenzethonium chloride; phenoctide; dodecarbonium chloride; and mixed alkyl-/acyl-, amidopropalkonium salts, or a mixture thereof.

The counter-ion can be any counter-ion that is pharmaceutically acceptable. Several such counter-ions are well known in the art. Some examples include, but not limited to, chloride, bromide, iodide, acetate, 2-ethylhexanoate, sulfate, phosphate, arylsulfonates, cyclohexylsulfamate, benzoate and saccharinate.

While a range of quaternary ammonium salt concentrations are suitable for this invention, in one aspect, the quaternary ammonium salt is present in a low concentration. In one aspect, this equals an amount of from about 0.1% to about 4.5% by weight of the pharmaceutically acceptable carrier. In another aspect of the invention, the quaternary ammonium salt may be present in an amount of from about 1% to about 4% by weight of the pharmaceutically acceptable carrier. In another aspect of the invention, the quaternary ammonium salt is present in an amount of about 1% by weight of the polymer. In yet another aspect of the invention, the said quaternary ammonium salt is present in an amount of about 2% by weight of the carrier.

### e) Synergism Aspects

In addition to acting as a penetration enhancer by itself, a quaternary ammonium salt may be combined with a second penetration enhancer substance (a co-enhancer) in order to achieve a synergistic result, which further increases the penetration enhancing effects of each enhancer.

Synergism is defined as a situation in which the combined effect of two agents is greater than that which would be predicted from their individual effects. For example, the agents may be skin permeation enhancers and the measured effect may be an increase in drug flux through the skin.

For the case in which both agents have some efficacy individually, the expected effect of a combination can be measured by using Loewe Additivity values (W. R. Greco et al. Pharmacological Reviews 47:331-385 (1995)).

The cumulative amount of drug permeating through the skin at time, t, is Qₜ (μg/cm²). For a system with Enhancer A at a concentration, aᵢ, the increase in flux relative to an unenhanced control (a=0) is defined as:

E(aᵢ)=[Qₜ(a=aᵢ)−Qₜ(a=0)]/Qₜ(a=0)=k_{A}aᵢ (1)

where k_{A} is a proportionality constant relating the concentration of Enhancer A to the flux increase. Using the Loewe Additivity Model, the expected effect of a combination of enhancers, A and B, is

É(aᵢ, bᵢ)=E(aᵢ)+E(bᵢ)=k_{A}aᵢ+k_{B}bᵢ. (2)

For a synergistic interaction between the two enhancers, the observed flux, E*(aᵢ, bᵢ), will be significantly greater than the expected effect and will be given by

E*(aᵢ, bᵢ)=É(aᵢ, bᵢ)+S=k_{A}aᵢ+k_{B}bᵢ.+S (3)

where S is a synergistic interaction term representing the part of the observed effect which is not predicted by the summation of the individual enhancer effects.

Using Equation 3 and assuming that k_{A} and k_{B} are constant over the concentration range of interest (i.e. assuming linearity), the expected effect of the combination of enhancers can be calculated from

É(aᵢ. bᵢ)=(a₂/a₁)E(a₁)+(b₂/b₁)E(b₁). (4)

The change in concentration of the individual enhancers, a₁→a₂ and b₁→b₂, was kept very small (typically from 0 to 10%). For these small changes in enhancer concentration, the linearity assumption has an almost insignificant effect on the calculated value of É(a₂, b₂).

The synergistic interaction term can then be calculated using Equation 4 and the actual observed effect of the combination of enhancers E*(a₂, b₂):

S=E*(a₂ b₂)−É(a₂ b₂). (5)

A synergistic interaction will be demonstrated when S has a significant positive value, meaning that the observed flux increase is substantially greater than would be expected from the combined effect of the individual enhancers.

In one aspect of the invention, the co-enhancer may be a compound represented by the formula:

R-Y

wherein R is a straight chain alkyl of about 7 to 17 carbon atoms, a non-terminal alkenyl of about 7 to 22 carbon atoms, or a branched-chain alkyl from about 12 to 22 carbons; and Y is —OH, —COOH, —OCOCH₃, —SOCH₃, —P(CH₃)₂O, —COO(C₂H₄O)ₘH, —(OC₂H₄)ₘOH, —COOCH₂CH(OH)CH₃, —COOCH₂CH(OH)CH₂OH, —COOCH₂CHXCH₂X, —CO(OCH₂CO)ₙOM, —CO[OCH(CH₃)CO]ₙOM —COOCH[CH(OH)]₄CH₂OH, —CO[C₆H₁₂O₆, sucrose], —CONR¹R², —COO(CH₂)₂NR¹R², —COO[CH(CH₃)CH₃]NR¹R², —COOR³, or N-pyrrolidone; where X is H or RCOO—; M is H or a pharmaceutically acceptable counter ion; R¹ and R² are independently H, CH₃, C₂H₅, C₃H₇, C₂H₄OH, or C₃H₇OH; R³ is CH₃, C₂H₅, or C₃H₇; m is an integer of 2 to 6; and n is an integer of 1 to 4. In some aspects, the co-enhancer is glycerol, or a glyceryl compound such as glyceryl monooleate, glyceryl dioleate, glyceryl trioleate, etc. In another aspect, the co-enhancer is triacetin.

In another aspect of the invention, the co-enhancer may be selected from the following group of agents: fatty acids and their salts, fatty alcohols, branched aliphatic alcohols, fatty acid alkyl esters (methyl, ethyl, isopropyl), fatty acid monoesters of sorbitol and glycerol, fatty acid esters with glycolic acid and lactylic acid and their salts, fatty acid amides (diethanolamides, monoethanolamides, and isopropanolamides), alkylpyrrolidones and mixtures thereof.

In yet another aspect of the invention, the co-enhancer may be selected from the following group of agents: oleic acid; lauric acid; oleyl alcohol; lauryl alcohol; 2-butyl-octanol; 2-hexyl decanol; 2-octyl-decanol; 2-hexyldodecanol; 2-octyl-dodecanol; 2-decyl-tetradecanol; 2-tetradecyl-octadecanol; methyl and ethyl laurate; sorbitan monooleate and monolaurate; glycerol monooleate and monolaurate; lauric, myristic, capric, stearic, and oleic diethanolamide; lauric, myristic, capric, stearic, and oleic monoethanolamide; lauric, myristic, capric, stearic, and oleic monoisopropanolamide; caproyl, lauroyl and stearoyl lactylic acid and their salts; caproyl, lauroyl and stearoyl glycolic acid and their salts; N-n-octyl and N-n-dodecyl pyrrolidone.

In one aspect, the synergism produces an enhancement of about 10% to about 100% or more. In another aspect, the enhancement is from about 10% to about 50%. In yet another aspect, the enhancement is from about 10% to about 20%. It is appreciated that various ranges of concentration of quaternary ammonium salts alone, or in combination with any of the co-enhancers described above, would result in various ranges of penetration enhancement. All such concentration ranges and ranges of enhancement are within the scope of the present invention.

### I) Irritation Reduction Aspects

In addition to acting as a penetration enhancer, the quaternary ammonium salt may also be present in an amount, which is sufficient to serve as an anti-irritant. Particularly, as shown in the examples below, quaternary ammonium salts are capable of retarding the growth of gram-negative, and gram-positive bacteria on the skin surface, underneath a transdermal drug delivery system. Skin irritation associated with transdermal patches and other occlusive devices has been attributed to increased bacterial growth on the skin surface underneath the transdermal patch. By retarding the growth and colonization of such bacteria, the accompanying skin irritation can be reduced.

It is generally known that quaternary an mnonium salts are irritating to the skin and thus have not been recommended as penetration enhancers. See, for example, Aoyagi, supra. While the quaternary ammonium salts are known to have some antimicrobial effects, they are not generally recommended for that purpose. For example, Remmington: The Science and Practice of Pharmacy, Vol. 2, pg. 1264-1265, 19^{th} ed. (1995) states:

The antiseptic [benzalkonium chloride] has slow action. It requires 7 min for the bacterial count on the skin to be decreased by a mere 50%, while only 36 seconds is required by 70% ethanol; to effect a 90% reduction, 25 minutes is required, compared to 2 minutes for 70% ethanol. Some gram-negative bacteria require hours of exposure to be killed . . . Like other cationic surface-active agents, has certain limitations. It does not destroy bacterial spores, it is ineffective against some viruses, it is inactivated by soap and other anionic surface-active agents, and when applied to the skin, it has a tendency to form a film under which bacteria remain viable. Organic matter from tissue inactivates [it], so that it has limited efficacy in the disinfection of wounds . . . [It] can cause irritation and damage the epidermis, and it also can cause allergies. In view of the availability of more reliable and more rapidly acting antiseptics, there is little to commend its continued use.

Thus, the use of quaternary ammonium salts is discouraged because they are slow or ineffective, irritating and allergenic.

Surprisingly, notwithstanding the above contrary teachings, the present inventors have discovered that low concentrations of quaternary ammonium salts can be effectively used in transdermal preparations not only to enhance penetration of a number of drugs, but also to reduce skin irritation associated with the application oftransdermal preparations. It is believed that, without wishing to be bound by any particular theory, the quaternary ammonium salts when used in such low concentrations have sufficient antimicrobial effect to prevent or retard microbial growth on the skin underneath the transdermal preparation and reduce irritation.

In one aspect, the low concentration of quaternary ammonium salt represents less than about 4.5% by weight of the carrier. In some aspects, the low concentration represents less than about 4.0% by weight of the carrier. In some aspects, the low concentration represents less than about 3.0% by weight of the carrier. In another aspect, the low concentration represents less than about 2.0% by weight of the carrier. In some other aspects, the low concentration represents less than about 1.0% by weight of the carrier. In some aspects the low concentration represents less than about 0.6% by weight of the carrier. In yet some aspects, the low concentration represents about 0.4% by weight of the carrier.

The microbials whose growth is controlled or retarded by the quaternary ammonium salts may be any bacteria, fungi or virus that is susceptible. In one aspect, the microbial may be gram-positive bacteria. In another aspect, the gram-positive bacteria may be gram-positive cocci. In some aspects, the microbials may be coagulase negative bacteria.

The skin irritation caused by the application of transdermal preparations may manifest in the form of erythema, papules, and vesicles. The present formulations comprising low concentrations of quaternary ammonium salts are effective in reducing these forms of irritation.

### g) Methods of Use and Administration

Further, methods for enhancing the transdermal penetration of a drug, reducing or preventing irritation associated with transdermal drug delivery, and providing a synergistic combination of penetration enhancers, are included in the present invention. Each of these methods comprises the step of combining a quaternary ammonium salt with a drug, and optionally a penetration enhancer for synergistic effects, and other ingredients as recited herein, into a carrier as recited herein, to form a transdermal drug delivery system, and administering such a system to a skin surface.

### h) Additional Aspects

While several aspects comprising a drug or a mixture of drugs for transdermal delivery have been described above, it is appreciated that the present invention can also be applied to provide topical formulations that do not comprise a drug. For example, due to the less irritating effects of the present invention, many applications can be envisioned wherein the formulations of the present invention can be used, with or without a topical drug (such as a topical antibiotic, topical anesthetic, a topical antihistamine, an anti-acne medication, etc.). When the formulation is provided without a drug, such formulation can be used simply as a wound-dressing composition, or a bandage to protect the site of a wound or other skin injury from the elements and microbials and help heal the affected skin faster. In such cases, the composition can be made occlusive (i.e., non-breathable) or non-occlusive (breathable), as needed. The methods for preparing occlusive and non-occlusive wound-dressing compositions are well known in the art. See for example, U.S. Pat. Nos. 3,949,128, 4,595,001, 4,798,201, 5,230,701, 5,246,705, 5,601,839, 5,713,842, 5,908,693, 5,626,866, 6,018,092, and 6,086,911, which are incorporated by reference.

### C. EXAMPLES

The following examples are intended to be merely illustrative of the various aspects of the invention disclosed herein and are not intended in any way to limit the scope of the claimed invention. Other aspects of the invention that are considered equivalent by those skilled in the art are also within the scope of this invention.

### Adhesive Matrix Preparation

The general procedures for preparing adhesive matrix patches are well known in the art. See. for example, U.S. Pat. Nos. 5,017,625, 5,234,957, 5,866,157, and 5,985,317, which are incorporated by reference. Pressure sensitive adhesives were obtained as solutions of adhesive polymers in organic solvents or as aqueous based emulsions. In order to prepare a drug-containing adhesive matrix film, the drug and other additives were first dissolved in the adhesive liquid, and then film coated and dried. Briefly, the procedure was as follows. The solid content of the adhesive solution was determined gravimetrically by evaporating the liquid phase from a known quality of adhesive. Measured amounts of the adhesive liquid were then mixed with appropriate quantities of drug and other excipients to yield the desired final dried film composition. In some cases, isopropanol was added to the adhesive mixture as a co-solvent to facilitate dissolution of the drug and/or excipients. The container with the adhesive and excipients was mixed on a rolling mill for 12-24 hours. These adhesive mixtures were coated and dried using either a small-scale bench-top procedure or a larger scale continuous coater/dryer in a pilot plant.

For the bench-top procedure, about 4 ml of the adhesive mixture was first dispensed onto a polyester liner with a silanized release coating (Coating A10/000 from Rexam Release Technologies; W. Chicago, Ill.). The mixture was then film cast with the appropriate gap-casting knife to achieve the desired dry coating thickness (typically 6 mg/cm²). The cast was dried in a convection oven at 70° C. for 15 minutes. After drying, an occlusive polyethylene backing film (Film 9720 from 3M Pharmaceuticals; St. Paul, Minn.) was laminated onto the adhesive. Patches or other samples were cut from these laminates using either a steel-rule die or a hole punch.

For the pilot plant coating and drying, the adhesive mixture was pumped through a slot die and continuously coated on release liner at 9 feet/minute. The coating was dried in a twelve-foot, two-zone convection oven at 100/120° C. The release liner and backing films used for the pilot plant coating were the same as in the bench-top coating. Patches were cut from these laminates using a rotary die. These matrix systems were then used to conduct wear study experiments as described below.

### Hydroalcoholic Gel Preparation

The general procedures for preparing hydroalcoholic gels are well known in the art. See, for example, U.S. Pat. Nos. 5,912,009, and 5,952,000 which are incorporated by reference. Hydroalcoholic gels were prepared by dissolving the drug and other additives in the appropriate hydroalcoholic solvent vehicle. When necessary, 2N NaOH was added to adjust the pH. The polymeric gelling agent was then added, and the mixture was mixed at least overnight on a rolling mill to form a viscous gel. The final pH of the gels was confirmed using an f100 ISFET pH meter (Beckman Instruments; Fullerton, Calif.) with 2-point calibration bracketing the range of interest.

### Permeation Enhancer Aspects

In vitro skin flux studies were conducted on epidermal membranes (stratum corneum and epidermis) obtained from whole human cadaver skin (epidermal membrane and dermis) by the heat-separation method of Kligman & Christopher, 88 Arch. Dermatol. 702 (1963). This method consists of immersion of the whole skin for 60 seconds in water at 60° C., followed by mechanical separation of the epidermal and dermal layers. After separation, the epidermal membrane is stored in aluminum foil at −5° C. until use.

Skin flux experiments were conducted in two-compartment glass diffusion cells with a modified Franz design. The receiver compartment was filled with water or an aqueous buffer appropriate to maintain sink conditions for the drug. All receiver media included 0.02% (w/w) sodium azide to inhibit bacterial growth.

For the measurement of skin flux from PSA matrix systems the adhesive matrix was affixed to the stratum corneum side of the thawed epidermal membrane and clamped between the two halves of the diffusion cell with the stratum corneum facing the donor compartment.

For the measurement of skin flux from hydroalcoholic gels, the thawed epidermal membrane was cut into rectangular strips and affixed to the diffusion cells with the stratum corneum side facing the donor compartment. A PTFE washer was placed on the donor side and 75 μl of gel was placed in the cavity at the center of the washer. The cavity was then covered with an occlusive backing film and clamped securely between the two halves of the diffusion cell.

During flux experiments, the diffusion cells were placed in a temperature-controlled circulating water bath calibrated to maintain the surface temperature of the skin at 32° C. The receiver compartment was continuously stirred with a magnetic stir-bar agitated by a stirring module placed under the water bath.

At predetermined sampling intervals, the entire volume of the receiver compartment solution was collected for drug quantification, and the receiver compartment was filled with fresh receiver solution, taking care to eliminate any air bubbles at the skin/solution interface.

Receiver solution samples were analyzed for drug content by HPLC with external standards of known drug concentration used for calibration. The cumulative amount of drug permeated per unit area at any time t (Qt, μg/cm²) was determined according to the following equation:

$${Q}_{t} = {\sum }_{n = 0}^{t} \frac{{C}_{n} V}{A}$$

where Ct (μg/cm³) is the concentration of the receiver compartment at sample time t (hours), V is the volume of the receiver compartment of the diffusion cell (6.3 cm³), and A is the diffusional area of the cell (0.64 cm²).

### Example 1

This example uses testosterone, a non-ionic androgenic steroid, as a model drug. Pressure-sensitive adhesive (PSA) transdermal patches were prepared using a medical grade acrylic/vinylpyrrolidone copolymer adhesive (DuroTak 87-2888; National Starch & Chemical, Bridgewater N.J.) according to the methods described above. The dried pressure sensitive adhesive matrix systems consisted of 6% (w/w) testosterone and 0 to 4% benzethonium chloride as an enhancer. The results of in vitro skin flux experiments using these matrix systems are summarized in Table 1.

TABLE 1
Effect of Benzethonium Chloride Concentration
on Testosterone Flux from a PSA Matrix
Composition: DuroTak-2888 Adhesive, 6% (w/w) Testosterone.

| | Q24 | |
|---|---|---|
| | μg/cm²/24 h, | |
| Benzethonium Chloride | Mean (SD), | |
| Concentration | n = 3, skins/15 cells | % Increase |
| 0% BzthCl | 33.1 (15.6) | 0% |
| 1% BzthCl | 39.3 (17.3) | 19% |
| 2% BzthCl | 46.8 (28.0) | 41% |
| 4% BzthCl | 62.2 (15.8) | 88% |

* Increase relative to the formulation containing 0% enhancer

These results demonstrate that benzethonium chloride increases the in vitro skin flux of testosterone from a pressure- sensitive adhesive matrix patch and that this increase is generally proportional to the concentration of benzethonium chloride in the patch.

### Example 2

In this example, the effect of benzethonium chloride on testosterone flux from a pressure-sensitive adhesive formulation, such as would be used in a matrix patch, is compared to its effect on testosterone flux from a hydroalcoholic gel, such as would be used for a liquid reservoir patch or a topical cream. Pressure-sensitive adhesive (PSA) transdermal patches were prepared using a medical grade acrylic/vinylpyrrolidone copolymer adhesive (DuroTak 87-2888) with a testosterone concentration of 6% (w/w) and benzethonium chloride concentrations of 0 and 1% (w/w). The hydroalcoholic gel vehicle consisted of 50% (v/v) ethanol, USP; 30% glycerin, NF; and 20% purified water, USP, gelled with 30 mg/ml hydrophobically modified carbomer (Permulen TR1 B F, Goodrich). Each gel was pH adjusted to a final pH 4±0.1 with 2N NaOH. Testosterone concentration in the gel vehicle was 1.5% (w/v) and the benzethonium chloride concentration was ranged from 0 to 1% (w/w). The results of in vitro skin flux experiments using these systems are summarized in Table 2.

TABLE 2
Effect of Benzethonium Chloride on Testosterone Flux
from an Acrylic PSA Matrix vs. a Hydroalcoholic Gel

| | | |
|---|---|---|
| | Q24 | |
| | μg/cm²/24 h | |
| Benzethonium Chloride | Mean (SEM), | |
| Concentration | n = 3 skins | % Increase |

DuroTak-2888 PSA Matrix with 6% (w/w) Testosterone

| | | |
|---|---|---|
| 0% BzthCl | 33.1 (9.0) | |
| 1% BzthCl | 39.3 (10.0) | 19% |

Hydroalcoholic Gel with 1.5% (w/v) Testosterone

| | | |
|---|---|---|
| 0% BzthCl | 44.4 (21.4) | |
| 1% BzthCl | 44.2 (18.0) | −0.5% |

These results show that a 1% concentration of benzethonium chloride in a hydroalcoholic gel formulation is insufficient as a penetration enhancer for testosterone. Surprisingly, these results also show that benzethonium chloride effectively increases the flux of testosterone from an adhesive matrix patch formulation.

### Example 3

This example uses oxybutynin hydrochloride, the salt form of a basic anticholinergic drug, as a model drug. In this example, the effect of benzethonium chloride on oxybutynin flux from a pressure sensitive adhesive matrix patch was compared to its effect on oxybutynin flux from a hydroalcoholic gel such as would be used for a liquid reservoir patch or a topical cream. Pressure-sensitive adhesive (PSA) transdermal patches were prepared using an aqueous emulsion polymerized acrylic copolymer adhesive (Morstick 214, Morton International) with an oxybutynin hydrochloride concentration of 5% (w/w) and benzethonium chloride concentrations of 0 and 1% (w/w). The hydroalcoholic gel vehicle consisted of a solvent composition of 50% (v/v) ethanol, USP; 30% (v/v) glycerin, NF; and 20% (v/v) purified water, USP. This solvent was gelled using 3% (w/v) modified hydroxyethyl cellulose (Natrosol Plus 330CS, Aqualon). Oxybutynin concentration in the gel vehicle was 5% (w/w) and the benzethonium chloride concentration was either 0 or 1% (w/w). Each gel was adjusted to a final pH of 5.00±0.05 using NaOH. Results of in vitro skin flux experiments using these systems are summarized in Table 3.

TABLE 3
Effect of Benzethonium Chloride on Oxybutynin Flux from an
Emulsion-Based Acrylic PSA Matrix vs. a Hydroalcoholic Gel

| | | |
|---|---|---|
| | Q24 | |
| | μg/cm²/24 h | |
| Benzethonium Chloride | Mean (SEM), | |
| Concentration | n = 3 skins | % Increase |

Morstick 214 PSA Matrix with 5% (w/w) Oxybutynin HCI

| | | |
|---|---|---|
| 0% BzthCl | 12.7 (2.5) | |
| 1% BzthCl | 18.9 (6.7) | 49% |

Hydroalcoholic Gel (pH 5) with 5% (w/w) Oxybutynin HCI

| | | |
|---|---|---|
| 0% BzthCl | 149.3 (77.0) | |
| 1% BzthCl | 64. (29.3) | −62% |

These results show that a 1% concentration of benzethonium chloride in a hydroalcoholic gel formulation is insufficient as a penetration enhancer for oxybutynin. Surprisingly, these results also show that benzethonium chloride effectively increases the flux of oxybutynin from an adhesive matrix patch formulation.

### Example 4

This example shows the skin flux enhancing effect of benzethonium chloride on the flux of a variety of model drugs from pressure-sensitive adhesive matrix patches. The pressure sensitive adhesives included 1) Duro-Tak 87-2888 (an organic solution-based acrylic/vinylpyrrolidone copolymer); 2) Duro-Tak 87-2979 (an organic solution-based acrylic); 3) Morstick 214 (an aqueous emulsion-based acrylic) and 4) Nacor 70-9965 (an aqueous emulsion-based acrylic). The model drugs tested were 1) estradiol (a non-ionic estrogen); 2) progesterone (a non-ionic progestin) and 3) buspirone (a basic anxiolytic) 4) propentofylline (a non-ionic xanthine derivative) and 5) oxybutynin (a basic anticholinergic drug). In each case, pressure-sensitive adhesive matrix patches were prepared at a constant drug concentration with and without benzethonium chloride. The results of in vitro skin flux experiments using these matrix systems are summarized in Table 4 and are reported in terms of percent increase in cumulative permeation relative to formulations containing no benzethonium chloride enhancer.

TABLE 4
Effect of Benzethonium Chloride on 24-hour Skin Flux
from PSA Patches Using a Variety of Model Drugs

| Pressure | | | | |
|---|---|---|---|---|
| Sensitive | | Benzethonium Chloride | Number of Skin | % Increase |
| Adhesive | Drug | Concentration % (w/w) | Sources | in Q24 |
| DuroTak-2888 | Buspirone | 1% BzthCl | 6 | 50 (14%) |
| | Estradiol | 1% BzthCl | 3 | 36 (9%) |
| | Oxybutynin | 1% BzthCl | 5 | 160 (60%) |
| | Progesterone | 1% BzthCl | 6 | 15 (9%) |
| | Propentofylline | 1% BzthCl | 3 | 32 (10%) |
| DuroTak-2979 | Estradiol | 0.5% BzthCl | 3 | 38 (15%) |
| Morstick 214 | Buspirone HCI | 1% BzthCl | 3 | 38 (21%) |
| Nacor-9965 | Oxybutynin HCI | 1% BzthCl | 3 | 24 (5%) |

* Increase relative to the formulation containing 0% enhance

These results demonstrate that benzethonium chloride increases the in vitro skin flux of a variety of model drugs from an adhesive formulation.

### Example 5

This example illustrates that the flux enhancing effect of benzalkonium chloride, another quaternary ammonium salt. This example uses testosterone as a model drug in a transdermal patch made from a medical grade acrylic/vinylpyrrolidone copolymer adhesive (Duro-Tak 87-2888, National Starch and Chemical). The dried pressure sensitive adhesive matrix systems consisted of 5% (w/w) testosterone and 0 or 2% benzalkonium chloride as an enhancer. The results of in vitro skin flux experiments using these matrix systems are summarized in Table 5.

TABLE 5
Effect of Benzalkonium Chloride on
Testosterone Flux from PSA Patches
Composition: 93-95% Duro-Tak 87-2888 Adhesive; 5% Testosterone

| | Q24 | |
|---|---|---|
| | μg/cm²/day | |
| Benzalkonium Chloride | Mean (SD) | |
| Concentration | n = 3 skins/15 cells | % Increase |
| 0% | 22.1 (6.0) | 0% |
| 2% | 45.6 (2.8) | 106% |

These results demonstrate that benzalkonium chloride increases the in vitro skin flux of testosterone from an adhesive formulation.

### Example 6

This example illustrates the flux enhancing effect of methylbenzethonium chloride usin g progesterone as a model drug in a PSA matrix system. The dried matrix systems consisted of Duro-Tak 87-2888 adhesive with 3% (w/w) progesterone and 0 or 0.5% methylbenzethonium chloride as an enhancer. The results of in vitro skin flux experiments using these matrix systems are summarized in Table 6.

TABLE 6
Effect of Methylbenzethonium Chloride
on Progesterone Flux from PSA Patches
Composition: DuroTak-2888 Adhesive, 3% (w/w) Progesterone

| | Q24 | |
|---|---|---|
| | μg/cm²/24 h, | |
| Methylbenzethonium | Mean (SD), | |
| Chloride Concentration | n = 3 skins/15 cells | % Increase |
| 0% | 32.9 (5.2) | 0.00 |
| 0.5% | 63.2 (6.9) | +92% |

These results demonstrate that methylbenzethonium chloride increases the in vitro skin flux of progesterone from an adhesive formulation.

### Example 7

This example illustrates that the flux enhancing effect of two quaternary ammonium salts: 1) olealkonium chloride (Incroquat O-50), and 2). N,N-diethyl-N-[2-[4-(1,1,3,3-tetramethylbutyl)phenoxy]ethyl]-benzenemethanaminium chloride (Phenoctide).

The model system for this example was a pressure sensitive adhesive matrix for the co-delivery of both estradiol and testosterone. The dried adhesive matrix consisted of Duro-Tak 87-2888 adhesive with 3.75% (w/w) testosterone and 8.5% (w/w) estradiol. A control system was prepared with no enhancer and enhanced systems were prepared with 2% (w/w) olealkonium chloride and phenoctide, respectively. The results of in vitro skin flux experiments using these matrix systems are summarized in Table 7.

TABLE 7
Effect of Olealkonium Chloride and Phenoctide on
Estradiol and Testosterone Flux from PSA Patches
Composition: DuroTak-2888 Adhesive, 3.5% (w/w) Testosterone, 8.5% Estradiol

| | Estradiol Flux | | Testosterone Flux | |
|---|---|---|---|---|
| | Q24 μg/cm²/24 h, | | Q24 μg/cm²/24 h, | |
| | Mean (SD), | | Mean (SD), | |
| Enhancer | n = 2 skins/10 cells | % Increase | n = 3 skins/15 cells | % Increase |
| 0% | 0.80 (0.07) | 0.00 | 9.07 (1.05) | 0.00 |
| 2% | 1.33 (0.04) | +66% | 13.54 (0.44) | +49% |
| Olealkonium | | | | |
| Chloride | | | | |
| 2% | 1.27 (0.13) | +59% | 13.00 (1.46) | +43% |
| Phenoctide | | | | |

These results demonstrate that olealkonium chloride and phenoctide increase the in vitro skin flux of estradiol and testosterone from a co-delivery matrix formulation.

These examples demonstrate that some quaternary ammonium salts, when used in low concentrations such as about less than 5%, are incapable of enhancing penetration of certain drugs such as testosterone in certain topical formulations such as hydroalcoholic gels. Surprisingly, these results also show that these quaternary ammonium salts are quite effective penetration enhancers even at such low doses when incorporated in an adhesive matrix patch formulation.

### Synergism Aspects

The following examples were conducted in accordance with the testing protocols recited above. However, it is appreciated that there might be some potential variability between skins from different individuals with respect to both total drug flux and enhancer effectiveness. Therefore the following systems were tested in parallel on each skin source:

1) An unenhanced control

2) A formulation with Enhancer A at concentration a₁

3) A formulation with Enhancer B at concentration b₁

4) A combined formulation with Enhancers A and B at concentrations a₂, b₂.

The concentrations of both enhancers in the combined system were restricted to be less than the concentration of either enhancer alone. This eliminates the possibility that the observed flux increase for the combination results merely from an unexpected effect of increasing the total enhancer concentration.

### Example 8

This example illustrates the effect of combining a quaternary ammonium salt such as benzethonium chloride (BzthCl), and a fatty acid glycerol ester such as glycerol monooleate (GMO), in a pressure sensitive adhesive matrix patch. The model drug is progesterone, a non-ionic steroid, and the adhesive used in the matrix system was DuroTak 87-2888, a vinylpyrrolidone/acrylic copolymer. The formulations prepared are described in Table 8:

TABLE 8
Matrix Patch Formulations

| | | Dry Composition |
|---|---|---|
| Formulation | Components | (% w/w) |
| Formulation 1 | DuroTak 87-2888 | 94% |
| Unenhanced | Progesterone | 6% |
| Formulation 2 | DuroTak 87-2888 | 93% |
| Benzyl | Progesterone | 6% |
| Ammonium Salt | Benzethonium Chloride | 1% |
| Only | | |
| Formulation 3 | DuroTak 87-2888 | 84% |
| Co-enhancer | Progesterone | 6% |
| Only | Glycerol Monooleate | 10% |
| Formulation 4 | DuroTak 87-2888 | 84% |
| Combination | Progesterone | 6% |
| | Benzethonium Chloride | 1% |
| | Glycerol Monooleate | 9% |

These formulations were evaluated using in vitro skin flux measurements on human cadaver skin and the results are presented in Table 9.

TABLE 9
Cumulative Progesterone Permeation In Vitro over 24 hours

| | | | | | | Formulation 4% | | | |
|---|---|---|---|---|---|---|---|---|---|
| | Form 1 | Formulation 2 | | Formulation 3% | | Combination | | Expected | Synergistic |
| | Unenhanced | 1% BzthCl | | 10% GMO | | 1% BzthCl/9% GMO | | Effect of | Interaction |
| | Q24 | Q24 | | Q24 | | Q24 | | Combination | Term |
| Skin Donor | (μg/cm²) | (μg/cm²) | E (%) | (μg/cm²) | E (%) | (μg/cm²) | E *(%) | E (%) | S (%) |
| Skin 1 | 11 6(3 3) | 13 2(12 7) | 14% | 15 8(4 3) | 36% | 44 2(33 9) | 281% | 46% | 235% |
| Skin 2 | 13 8(3 0) | 14 7(2 8) | 7% | 13 1(3 0) | −5% | 49 4(3 6) | 258% | 2% | 256% |
| Skin 3 | 24 5(0 4) | 29 3(1 4) | 20% | 39 6(1 8) | 62% | 71 4(16 6) | 191% | 75% | 116% |
| Mean (SEM) | 16 6(4 0) | 91 1(5 1) | 14(4)% | 22 8(8 9) | 31(19)% | 55 0(14 4) | 243(27)% | 44(21)% | 200(43)% |
| All Skins | | | | | | | | | |

In this example, both benzethonium chloride and glycerol monooleate had a measurable effect on progesterone skin flux. Using the Loewe Additivity Model, one can calculate the expected effect from the combination of these two enhancers. For example, using the data for Skin 1, the expected flux increase for the combined enhancers using Equation 4 is:

É(1% BzthCl, 9% GMO)=(1/1)*14%+(9/10)*31%=42%

The actual flux increase for the combined enhancers was 243%, which is nearly six times greater than the expected value and results in a synergistic interaction term of>200%.

The average synergistic interaction term for the three independent skin sources was Mean(SEM)=200(43)%, illustrating that there is a strong and consistent synergism when benzethonium chloride and glycerol monooleate are combined in a matrix patch.

### Example 9

This example illustrates that synergism is observed between a quaternary ammonium compound and a variety of co-enhancers. For this example, progesterone was used as a model drug and benzethonium chloride (BzthCl) was used as a model quaternary ammonium compound. The representative co-enhancers tested are summarized in Table 10.

TABLE 10
Characteristics of Some Co-Enhancers

| | | Predominant | |
|---|---|---|---|
| | | Hydrophobic | Polar Head |
| Co-Enhancer | Trademark | Chain | Group |
| 2-Butyl-octanol | Isofol ® 12 | C12 (branched) | Alcohol |
| Lauric Acid | Monamid ® | C12 | Alkanolamide |
| Diethanolamide | 150LWA | | ester |
| Lauric Acid | Alkamide ® | C12 | Alkanolamide |
| Monoisopropanol- | LIPA | | ester |
| amide | | | |
| Lauryl Alcohol | EPAL ® 12 | C12 | Alcohol |
| Oleic Acid | Oleic Acid, NF | C18 (unsaturated) | Acid |
| Oleic Acid | Alkamide ® | C18 (unsaturated) | Alkanolamide |
| Diethanolamide | DO-280 | | ester |
| Oleyl Alcohol | Oleyl Alcohol | C18 (unsaturated) | Alcohol |
| Sorbitan | Arlacel ® 80 | C18 (unsaturated) | Sorbitol Ester |
| Monooleate | | | |
| Stearic Acid | Alkamide ® | C18 | Alkanolamide |
| Diethanolamide | DS-280 | | ester |
| N-n-octyl- | Surfadone ® | C8 | Pyrrolidone |
| pyrrolidone | LP-100 | | |

For each co-enhancer, the following four drug-in-adhesive matrix formulations were prepared: I) Formulation with no enhancer, II) Formulation with benzethonium chloride only, III) Formulation with the co-enhancer only, IV) Formulation with a combination of benzethonium chloride and the co-enhancer combined. All formulations used DuroTak 87-2888 pressure sensitive adhesive. Details of the formulations tested are listed in Table 11.

TABLE 11
Composition of Progesterone Matrix Formulations Tested
Compositions: All formulations were prepared in DuroTak 87-2888 Adhesive

| | | Progesterone | Enhancer Composition | | | |
|---|---|---|---|---|---|---|
| Example | Co-enhancer | Concentration | Form I | Form II | Form III | Form IV |
| 2-A | 2-butyl- | 3% | No | 10.0% | 0.5% | 9.5% CoEnh/ |
| | octanol | | Enhancers | CoEnh | BzthCl | 0.5% BzthCl |
| 2-B | Lauric Acid | 3% | No | 10.0% | 0.4% | 9.6% CoEnh/ |
| | Diethanolamide | | Enhancers | CoEnh | BzthCl | 0.4% BzthCl |
| 2-C | Lauric Acid | 3% | No | 10.0% | 0.4% | 9.6% Coenh/ |
| | Monoiso- | | Enhancers | CoEnh | BZthCl | 0.4% BzthCl |
| | propanolamide | | | | | |
| 2-D | Lauryl | 3% | No | 10.0% | 0.4% | 9.6% CoEnh/ |
| | Alcohol | | Enhancers | CoEnh | BzthCl | 0.4% BzthCl |
| 2-E | Oleic Acid | 3% | No | 10.0% | 0.4% | 9.6% CoEnh/ |
| | | | Enhancers | CoEnh | BzthCl | 0.4% BzthCl |
| 2-F | Oleic Acid | 3% | No | 10.0% | 0.4% | 9.6% CoEnh/ |
| | Diethanolamide | | Enhancers | CoEnh | BzthCl | 0.4% BzthCl |
| 2-G | Oleyl | 3% | No | 10.0% | 0.4% | 9.6% CoEnh/ |
| | Alcohol | | Enhancers | CoEnh | BzthCl | 0.4% BzthCl |
| 2-H | Sorbitan | 6% | No | 10.0% | 1.0% | 9.0% Coenh/ |
| | Monooleate | | Enhancers | CoEnh | BzthCl | 1.0% BzthCl |
| 2-I | Stearic Acid | 3% | No | 10.0% | 0.4% | 9.6% CoEnh/ |
| | Diethanolamide | | Enhancers | CoEnh | BzthCl | 0.4% BzthCl |
| 2-J | N-n-octyl- | 3% | No | 10.0% | 1.0% | 9.0% CoEnh/ |
| | pyrrolidone | | Enhancers | CoEnh | BzthCl | 1.0% BzthCl |

In vitro skin flux studies were conducted on these formulations on skin from three skin donors over a 24-hour period. Enhancement factors were determined by comparing the cumulative drug flux from Formulations II-IV with the cumulative flux from Formulation I (no enhancer) on the same skin donor. These enhancement factors were then used to calculate the degree of synergism as described in Example 8. The results of these experiments are summarized in Table 12.

TABLE 12
Results of In Vitro Skin Flux Testing of Progeterone Matrix Formulations
Mean (SEM), n = 3 skin donors

| | | | Enhancement | | | |
|---|---|---|---|---|---|---|
| | | Enhancement | Factor, | Enhancement | Expected | Synergistic |
| | | Factor, | Co-enhancer | Factor, | Effect of | Interaction |
| | | BzthCl Only | Only | Combination | Combination | Term |
| Example | Co-enhancer | E₁ | E₂ | E* | É | S |
| 2-A | 2-butyl-octanol | 32 (6)% | 116 (29)% | 161 (38)% | 142 (22)% | 19 (27)% |
| 2-B | Lauric Acid | 20 (9)% | 23 (5)% | 109 (69)% | 42 (12)% | 66 (57)% |
| | Diethanolamide | | | | | |
| 2-C | Laurie Acid | 3 (7)% | 11 (2)% | 50 (14)% | 13 (6)% | 37 (13)% |
| | Monoisopropano | | | | | |
| | lamide | | | | | |
| 2-D | Lauryl Alcohol | 7 (1)% | 41 (11)% | 137 (17)% | 47 (11)% | 90 (6)% |
| 2-E | Oleic Acid | 7 (7)% | 26 (15)% | 82 (16)% | 32 (16)% | 51 (1)% |
| 2-F | Oleic Acid | 11 (15)% | 16 (7)% | 47 (7)% | 27 (21)% | 20 (27)% |
| | Diethanolamide | | | | | |
| 2-G | Oleyl Alcohol | 19 (8)% | 22 (13)% | 83 (8)% | 39 (19)% | 44 (22)% |
| 2-H | Sorbitan | 22 (5)% | 54 (13)% | 141 (63)% | 70 (15)% | 71 (49)% |
| | Monooleate | | | | | |
| 2-I | Stearic Acid | 7 (7)% | 26 (15)% | 82 (16)% | 31 (16)% | 51 (0)% |
| | Diethanolamide | | | | | |
| 2-J | N-n-octyl- | 30 (5)% | 36 (8)% | 169 (27)% | 63 (5)% | 107 (28)% |
| | pyrrolidinone | | | | | |

For all the formulations tested, the flux for the formulation containing the combination of benzethonium chloride and the co-enhancer was from ˜20-100% greater than would be expected assuming an additive combination of the enhancing effects of benzethonium chloride and the co-enhancer. These results confirm the synergistic effect of combining benzethonium chloride with a co-enhancer.

### Example 10

This example illustrates that synergism is observed between quaternary ammonium compounds and co-enhancers using model drugs such as: 1) testosterone, an androgenic steroid; 2) estradiol, an estrogenic steroid, and 3) buspirone, an anxiolytic. Benzethonium chloride was used as a model ammonium compound, and the co-enhancers were sorbitan monooleate. lauric acid diethanolamide, and caproyl lactylic acid (Pationic CLA). All formulations were drug-in-adhesive matrix patches prepared in DuroTak 87-2888 pressure sensitive adhesive. Details of the formulation compositions are shown in Table 13.

TABLE 13
Composition of Steroid Matrix Formulations Tested
Compositions: All Formulations Were Prepared in DuroTak 87-2888 Adhesive

| | | | Enhancer Composition | | | |
|---|---|---|---|---|---|---|
| Example | Co-enhancer | Drug | Form I | Form II | Form III | Form IV |
| 3-A | Lauric Acid | 8% Estradiol | No | 10.0% | 0.4% BzthCl | 9.6% CoEnh/ |
| | Diethanolamide | | Enhancers | CoEnh | | 0.4% BzthCl |
| 3-B | Sorbitan | 6% | No | 7.5% CoEnh | 1.0% BzthCl | 6.5% CoEnh/ |
| | Monooleate | Testosterone | Enhancers | | | 1.0% BzthCl |
| 3-C | Caproyl Lactylic | 2% | No | 2.0% Co-Enh | 2.0% BzthCl | 1.0% Coenh./ |
| | Acid | Buspirone | Enhancers | | | 1.0% BzthCl |

In vitro skin flux studies were conducted on these formulations over 24 hours, and the results are summarized in Table 14.

TABLE 14
Results of In Vitro Flux Testing of Matrix Formulations
Mean (SEM), n = 3 skin donors

| | | | | Enhancement | | | |
|---|---|---|---|---|---|---|---|
| | | | Enhancement | Factor, | Enhancement | | Synergistic |
| | | | Factor, | Co-enhancer | Factor, | Expected Effect | Interaction |
| | | | BzthCl Only | Only | Combination | of Combination | Term |
| Example | Drug | Co-enhancer | E₁ | E₂ | E* | É | S |
| 3-A | Testosterone | Lauric Acid | 17 (3)% | 87 (14)% | 140 (20)% | 93 (9)% | 47 (23)% |
| | | Diethan- | | | | | |
| | | olamide | | | | | |
| 3-B | Estradiol | Sorbitan | 36 (9)% | 22 (7)% | 93 (6)% | 55 (13)% | 37 (8)% |
| | | Monooleate | | | | | |
| 3-C | Buspirone | Caproyl | 12 (9)% | 12 (9)% | 12 (4)% | 12 (4)% | 10 (27)% |
| | | Lactylic | | | | | |
| | | Acid | | | | | |

Flux for the formulations containing the combination of benzethonium chloride and the co-enhancer was about 10-50% greater than would be expected assuming an additive combination of the enhancing effects of benzethonium chloride and the co-enhancer. These results confirm the synergistic effect of combining benzethonium chloride with a co-enhancer for a variety of drugs such as estradiol, testosterone, and buspirone.

### Anti-Irritant Aspects

Polymeric adhesive formulations were made in accordance with the above-recited protocol for testing to determine the value of quaternary ammonium salts as anti-irritants. The following examples illustrate the anti-irritant properties imparted to a transdermal drug delivery system by the inclusion of a quaternary ammonium salt in accordance with the present invention.

### Example 11

Placebo transdermal matrix patches were manufactured and worn in a wear study. The patches were 10 cm² in size with matrix compositions of pressure sensitive acrylic/polyvinylpyrrolidone copolymer adhesive (TSR 58, Sekisui Chemical Company) and 10% w/w of a proprietary skin permeation enhancer, sorbitan monooleate (Arlacel 80, ICI Americas).

Two subjects wore Patches on the arm for a 96-hour application period. After removal the application sites were evaluated for local skin reaction. One subject exhibited an unusually severe adverse skin reaction (erythema and papules). The second subject exhibited no significant skin reaction.

These patches were subjected to a microbiological investigation to determine whether there was any difference in the microbial growth under these patches. Unopened, unused placebo patches and worn patches from these two individuals were examined for microbiological bioburden by briefly contacting the adhesive surface to a plate of Trypticase Soy Agar (Soybean Casein Digest Agar; USP 23:61, Medium II), a general purpose supportive medium for microbial growth. The plates were then incubated overnight at 32.5° C. and examined at 10× magnification for microbial growth. Microbial colonies were further identified by staining and examination at 1,000×. Results of this investigation were as follows:

1) Unused patches, which had never been worn, exhibited no microbial growth in this test.

2) The patch from the individual with no adverse skin reaction showed minimal microbial growth.

The patch from the individual with a strong adverse skin reaction exhibited extensive, confluent overgrowth of exclusively gram-positive cocci. These were found to be coagulase-negative using the Coagulase Test for Staphylococcus aureus (USP 23:61) (designated as E3 herein).

These results suggest that local skin irritation resulting from wearing transdermal patches may in some cases be associated with microbial overgrowth, and more specifically, with overgrowth of gram-positive, coagulase-negative cocci.

### Example 12

The effect of microbial growth on skin reactions from matrix patches was investigated in a larger population by conducting an experiment in which volunteers wore two placebo matrix patches on abdominal sites--one control on an untreated skin site and one on a site, which had been swabbed with an isopropanol-saturated pad just prior to application. The patches were 10 cm² pressure sensitive adhesive matrix patches consisting of TSR 58 adhesive and 10% w/w sorbitan monooleate.

Eighteen subjects wore the patches for a 96-hour application period. The skin reactions at the sites were evaluated at 1 hour and 24 hours after patch removal by trained observers. Skin reaction was scored with respect to degree of erythema (DE) using the following scale:

0=none

1=mild (faint or barely perceptible)

2=moderate (bright pink or sunburned appearance)

3=severe (beet red)

The presence of other skin reactions (e.g. edema, papules, and vesicles) was also recorded. Results of the skin reaction observations at one hour after patch removal and 24 hours after patch removal are summarized in Tables 15 and 16, respectively.

TABLE 15
Effect of Alcohol Wiping on Skin Toleration of
Matrix Patches (1 hour after Patch Removal)
Observations at 1 Hour Post Removal
Number of Subjects with Observed Reaction
(Percentage of Subjects in Parentheses)

| | Degree of Erythema | | | |
|---|---|---|---|---|
| | None | Mild | Moderate | Other Skin |
| Patch Site | DE = 0 | DE = 1 | DE = 2 | Reactions |
| Untreated Site | 4 (22%) | 10 (56%) | 4 (22%) | 3 (13%) |
| (Control) | | | | [papules |
| Alcohol Wiped | 13 (72%) | 5 (28%) | 0 (0%) | 1 (6%) |
| Site | | | | [papules] |

TABLE 16
Effect of Alcohol Wiping on Skin Toleration of
Matrix Patches (24 hours after Patch Removal)
Observations at 24 Hours Post Removal
Number of Subjects with Observed Reaction
(Percentage of subjects in parentheses)

| | Degree of Erythema | | | |
|---|---|---|---|---|
| | None | Mild | Moderate | |
| Patch Site | DE = 0 | DE = 1 | DE = 2 | Other Skin Reactions |
| Untreated Site | 4 (22%) | 6 (33%) | 8 (44%) | 2 (13%) |
| (Control) | | | | [papules] |
| Alcohol Wiped | 12 (67%) | 4 (22%) | 2 (11%) | 0 (0%) |
| Site | | | | [papules] |

The scores at one hour after patch removal show that wiping the site with alcohol decreased the incidence of mild and moderate erythema substantially. The number of subjects exhibiting no erythema increased from 22% for the control patch to 72% for the patch at the alcohol wiped site. Incidence of papules at the patch application site was also reduced from 13% for the control to 6% for the alcohol wiped site. Similar trends were seen at 24 hours after patch removal. These results show that wiping the skin site with alcohol prior to patch application significantly reduces the irritation and other adverse skin reactions from transdermal matrix patches.

### Microbiological Analysis

Patches were removed, covered with a silicone release liner and stored pouched and refrigerated overnight at 4° C. The patches were returned to room temperature, then under aseptic conditions the release liner was removed and the adhesive surface was pressed briefly onto the surface of a agar plate. Eight of the eighteen patches were cultured on a general purpose medium—Trypticase Soy Agar (TSA), and nine of the eighteen patches were cultured on a medium specific for yeasts and molds—Potato Dextrose Agar (PDA) (USP 23:61, Medium XX). The inoculated plates were incubated at 32.5□ C. for eighteen hours for the TSA and 6 days for PDA. The plates were examined at 10× magnification by an individual who was blinded to the composition of the patches and scored bacterial growth using the following scale:

1=Minimal growth

2=Significant growth

3=Total overrun of patch area (confluent)

Microbial morphology was determined by staining and examination (1,000× magnification) of the cultures. Results of this scoring for the Trypticase Soy Agar culture are summarized in Table 17.

TABLE 17
Effect of Alcohol Wiping on Microbial
Growth Under Matrix Patches
Microbial Growth in TSA
Number of Patches with
Observed Microbial Growth Score
(Percentage of Patches in Parentheses)

| | Microbial Growth Score | | |
|---|---|---|---|
| | Minimal | Significant | Overrun |
| Patch Site | Score = 1 | Score = 2 | Score = 3 |
| Untreated Site | 0 (0%) | 1 (12.5%) | 7 (87.5%) |
| (Control) | | | |
| Alcohol Wiped | 6 (75%) | 2 (25%) | 0 (0%) |
| Site | | | |

Patches from the control site without the alcohol wipe showed extensive microbial growth of almost exclusively gram-positive, coagulase-negative cocci. Patches at the alcohol-wiped site showed significantly reduced microbial growth in all cases. These results, together with the skin reaction observations, support the hypothesis that there is an association between microbial growth under the patch surface and observed skin reactions such as erythema and papules.

The cultures with PDA grew no yeasts or molds and, with only one exception, exhibited relatively little bacterial growth for both the control and alcohol wiped sites. The exception was a control patch from an untreated site, which was overrun with gram-negative coccobacilli. No erythema or other skin reaction was observed at the site where this patch was worn.

These results further indicate that skin irritation and other adverse skin reactions may be associated specifically with bacterial overgrowth under the patch surface, and more specifically with overgrowth of gram-positive, coagulase-negative cocci.

### Example 13

In this experiment, the effectiveness of various topical antimicrobial agents against the E3 organism was determined using Zone of Inhibition testing of paper discs saturated with aqueous antimicrobial solutions. The results of these in vitro tests are shown in Table 18.

TABLE 18
In Vitro Testing for Antimicrobial Efficacy of Aqueous Solutions
against E3 Organism Aqueous Solutions Loaded on 0.33 cm² Paper
Disks, (8.5 ± 0.5 mg Solution/Disc)

| | Antimicrobial | Zone of |
|---|---|---|
| | Concentration | Inhibition |
| Compound | (% w/w) | (mm) |
| Water (Control) | 0 | 0 |
| Benzalkonium Chloride | 0.4 | 13 |
| Benzethonium Chloride | 0.4 | 12 |
| Benzoic Acid | 2.0 | 10 |
| Benzyl Alcohol | 2.0 | 10 |
| Methyl Paraben | 2.0 | 0 |

Of the antimicrobial agents tested, the two quaternary ammonium salts, benzethonium chloride and benzalkonium chloride, were the most effective. Benzoic acid and benzyl alcohol also showed some activity against E3, when present at significantly higher concentrations.

### Example 14

Having identified antimicrobial candidates which were effective in aqueous solution against the E3 gram positive cocci isolate, the next step was to determine whether the same antimicrobial agents would be effective when incorporated in a transdermal matrix patch. Transdermal matrix patches were prepared containing 0.4% w/w benzalkonium chloride, benzethonium chloride, or benzoic acid in a pressure sensitive adhesive matrix (DuroTak 87-2888 adhesive). These patches were cut into 1 cm² disks and subjected to Zone of Inhibition testing against the E3 organism with 24-hour incubation. The results of these tests are summarized in Table 19.

TABLE 19
In Vitro Testing for Antimicrobial Efficacy of Antimicrobial-Loaded
Transdermal Matrix Samples Against E3 Organism Adhesive Matrix
Disks, 1.1 cm² Area, (7.8 ± 1.0 mg Adhesive Matrix-Disc)

| | Antimicrobial | Zone of |
|---|---|---|
| Antimicrobial | Concentration | Inhibition |
| Compound | (% w/w) | (mm) |
| (Control) | 0 | <0* |
| Adhesive Only | | |
| Benzalkonium Chloride | 0.4 | 15 |
| Benzethonium Chloride | 0.4 | 17 |
| Benzoic Acid | 0.4 | <0 |

*Indicates that microbial growth occurred under the sample.

Among the antimicrobial agents identified as effective in Example 12, the quaternary ammonium salts, benzethonium chloride and benzalkonium chloride, were particularly effective when incorporated into a transdermal matrix formulation.

### Example 15

In the next experiment, placebo matrix patches (18 cm² area) were manufactured for a clinical wear study. A control consisted of DuroTak 87-2888 adhesive and 10% w/w of sorbitan monooleate. Test patches consisted of DuroTak 87-2888 adhesive, 10% sorbitan monooleate, and 0.4% benzethonium chloride (BzthCl). In vitro Zone of Inhibition testing against E3 was conducted on these patches as described in Example 14. The zone of inhibition was <0 mm for the control patch and 26 mm for the test patch with 0.4% BzthCl, consistent with the results in Example 14.

A clinical wear study was conducted on 16 volunteers who wore each patch on the abdomen a t randomized sites for 96 hours. After removal of the patches, the skin reaction at the sites was evaluated at 1 hour and 24 hours after removal by trained observers who were blinded as to the composition of the patches.

Skin reaction was scored with respect to degree of erythema (DE) using the following scale:

0=none

1=mild (faint or barely perceptible)

2=moderate (bright pink or sunburned appearance)

3=severe (beet red)

The presence of other skin reactions (e.g. edema, papules, and vesicles) was also recorded. Results of the skin reaction observations at one hour after patch removal and 24 hours after patch removal are summarized in Tables 20 and 21, respectively.

TABLE 20
Effect of Benzethonium Chloride on Skin Toleration of Matrix Patches
(1 Hour After Patch Removal)
Observation at 1 Hour Post Removal
Number of Subjects with Observed Reaction
(Percentage of Subjects in Parenteses)

| | Degree of Erythema | | | | |
|---|---|---|---|---|---|
| | None | Mild | Moderate | Severe | Other Skin |
| Matrix Patch | DE = 0 | DE = 1 | DE = 2 | DE = 3 | Reactions |
| No BzthCl | 3 (19%) | 8 (50%) | 4 (25%) | 1 (6%) | 2 (13%) |
| (Control) | | | | | [papules] |
| 0.4% BzthCl | 13 (81%) | 3 (19%) | 0 (0%) | 0 (0%) | 0 (0%) |

TABLE 21
Effect of Benzethonium Chloride on Skin Toleration of Matrix Patches
(24 Hours After Patch Removal)
Observations at 24 Hour Post Removal
Number of Subjects with Observed Reaction
(Percentage of Subjects in Parenteses)¹

| | Degree of Erythema | | | | |
|---|---|---|---|---|---|
| | None | Mild | Moderate | Severe | Other Skin |
| Matrix Patch | DE = 0 | DE = 1 | DE = 2 | DE= 3 | Reactions |
| 0% BzthCl | 3 (21.%) | 7 (50%) | 3 (21%) | 1 (7%) | 0 (0%) |
| 0.4% BzthCl | 13 (93%) | 1 (7%) | 0 (0%) | 0 (0%) | 0 (0%) |

¹Only 14 of the 16 subjects were evaluated at 24 hours post-removal.

These wear study results show that at 1 hour after patch removal the incidence of mild to severe erythema at the control patch site was 78.5%, while the incidence at the site of the patch containing BzthCl was only 19% (all mild). Papules were observed in two subjects at the control patch site (with coincident erythema scores of 1 and 3). In both subjects, the site of the patch containing benzethonium chloride exhibited no evidence of papules and no erythema.

Similar trends were seen at 24 hours after patch removal, with 79% of the subjects exhibiting mild to severe irritation at the control patch site and only one out of 14 (7%) of the subjects exhibiting any erythema (mild) at the site of the patch containing 0.4% benzethonium chloride.

These results show that the addition of an antimicrobial agent with a narrow spectrum of activity against gram-positive cocci can drastically reduce skin irritation associated with patch application. Surprisingly, this effect is not limited to those individuals with particularly strong irritation responses (moderate to severe erythema and/or papules), but is seen to occur broadly across all subjects.

These examples demonstrate how benzethonium chloride, as representative of quaternary amine antimicrobials reduces skin irritation associated with application of a transdermal drug delivery device when incorporated therein.

What is claimed is:

1. A transdermal composition comprising a pharmaceutically acceptable carrier, a drug, and a quaternary ammonium salt constituting from about 0.1% to about 4.5% by weight of the carrier.

2. The transdermal composition of claim 1, wherein said quaternary ammonium salt is a compound having the formula: 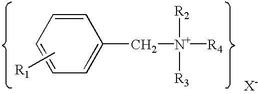
wherein R₁ is a member selected from the group consisting of H and C₁-C₁₂ straight or branched chain alkyl; R₂ and R₃ are independent members selected from the group consisting of CH₃, —CH₂OH and —CH₂ CH₂OH; R₄ is a member selected from the group consisting of:
(a) CH₃,
(b) C₂-C₂₂ straight or branched chain alkyl,
(c) C₂-C₂₂ straight or branched chain alkenyl,
(d) [CH₂CH₂O]ₙ—R₅ where n is an integer of 1-3 and R₅ is a member selected from the group consisting of H, C₁-C₁₂ straight or branched chain alkyl, C₂ - C₂₂ straight or branched alkenyl; and 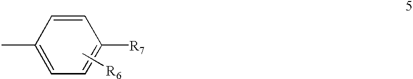
wherein R₆ is a member selected from the group consisting of H and —CH₃ and R₇ is a member selected from the group consisting of C₁-C₂₂ straight or branched chain alkyl and C₂-C₂₂ straight or branched chain alkenyl, and
(e)—(CH₂)ₘ NOCR₇ or —(CH₂)ₘ CONR₇ where m is an integer of 1-3; and
X is a pharmaceuticaly acceptable counter-ion.

3. The transdermal composition of claim 1, wherein said quaternary ammonium salt is benzalkonium chloride; benzalkonium saccharinate; behenalkonium chloride; cetalkonium chloride; erucalkonium chloride; lauralkonium chloride; myristalkonium chloride; myristalkonium saccharinate (Quaternium-3); stearalkonium chloride; olealkonium chloride; tallowalkonium chloride; dodecylbenzyltrimethylammonium chloride (Quaternium-28); dodecylbenzyl trimethyl ammonium 2-ethylhexanoate; ethylbenzyl alkyldimethylammonium cyclohexylsulfanamate (Quaternium-8); ethylbenzyl dimethyl dodecyl ammonium chloride (Quaternium-14); dodecylbenzyl dimethyl octadecyl ammonium chloride; dodecylbenzyl triethanol ammonium chloride (Quaternium-30); benzoxonium chloride; benzylbis(2-hydroxyethyl)(2-dodecyloxyethyl)ammonium bromide; benzylbis(2-hydroxyethyl)(2-dodecyloxyethyl)ammonium chloride; benzethonium chloride; methylbenzethonium chloride; N,N-(diethyl-N-[2-[4-(1,1,3,3-tetramethylbutyl)phenoxy]ethyl] benzenemethanaminium chloride (phenoctide); dodecarbonium chloride; babassuamidopropalkonium chloride; wheatgermamidopropalkonium chloride, or a mixture thereof.

4. The transdermal composition of claim 1, wherein said quaternary ammonium salt is benzalkonium chloride, stearalkonium, behenalkonium chloride, olealkonium chloride, erucalkonium chloride, benzethonium chloride, methylbenzethonium chloride, phenoctide, wheatgermamidopropalkonium chloride, babassuamidopropalkonium chloride or a mixture thereof.

5. The transdermal composition of claim 1, wherein the quaternary ammonium salt is benzethonium chloride.

6. The transdermal composition of claim 1, wherein the quaternary ammonium salt is methylbenzethonium chloride.

7. The transdermal composition of claim 1, wherein the quaternary ammonium salt is benzalkonium chloride.

8. The transdermal composition of claim 1, wherein the quaternary ammonium salt is olealkonium chloride.

9. The transdermal composition of claim 1, wherein the quaternary ammonium salt is phenoctide.

10. The transdermal composition of claim 2, wherein the quaternary ammonium salt is present in an amount sufficient to act as an anti-irritant.

11. The transdermal composition of claim 10, wherein said quaternary ammonium salt is a member selected from the group consisting of alkyl-, dimethyl benzenemethanaminium salts; acyl-, dimethyl benzenemethanaminium salts; mixed acyl-/alkyl-, dimethyl benzenemethanaminium salts; ethylbenzyl dodecyl dimethylammonium chloride, dodecylbenzyltrimethylammonium chloride, dodecylbenzyl triethanolammonium chloride, benzoxonium chloride, benzethonium chloride; methylbenzethonium chloride; phenoctide; dodecarbonium chloride; and mixed alkyl-/acyl-, amidopropalkonium salts.

12. The transdermal composition of claim 1, wherein said quaternary ammonium salt constitutes about 1% by weight of the pharmaceutically acceptable carrier.

13. The transdermal composition of claim 1, wherein said quaternary ammonium salt constitutes about 2% by weight of the pharmaceutically acceptable carrier.

14. The transdermal composition of claim 1, wherein said pharmaceutically acceptable carrier is a biocompatible polymer.

15. The transdermal composition of claim 1, wherein said pharmaceutically acceptable carrier is an adhesive.

16. The transdermal composition of claim 15, wherein said adhesive is a member selected from the group consisting of acrylics, vinyl acetates, natural and synthetic rubbers, ethylene-vinyl acetate copolymers, polysiloxanes, polyacrylates, polyurethanes, plasticized polyether block amide copolymers, plasticized styrene-rubber block copolymers, and mixtures thereof.

17. The transdermal composition of claim 1, wherein said pharmaceutically acceptable carrier comprises a viscous material suitable for inclusion in a liquid reservoir.

18. The transdermal composition of claim 17, wherein said viscous material forms a gel.

19. The transdermal composition of claim 2, wherein the counter-ion is selected from the group consisting of chloride, bromide, iodide, acetate, 2-ethylhexanoate, sulfate, phosphate. arylsulfonates, cyclohexylsulfamate, benzoate, saccharinate, and a mixture thereof.

20. The transdermal composition of claim 1, further comprising a diluent, excipient, emollient, plasticizer, skin irritation reducing agent, or a mixture thereof.

21. The transdermal composition of claim 1, further comprising a co-enhancer that acts synergistically with the quaternary ammonium salt to enhance the penetration of the drug.

22. The transdermal composition of claim 21, wherein said co-enhancer comprises a compound represented by the formula:
R-Y
wherein R is a straight chain alkyl of about 7 to 17 carbon atoms, a non-terminal alkenyl of about 7 to 22 carbon atoms, or a branched-chain alkyl from about 12 to 22 carbons; and Y is —OH, —COOH, —OCOCH₃, —SOCH₃, —P(CH₃)₂O, —COO(C₂H₄O)ₘH, —(OC₂H₄)ₘOH, —COOCH₂CH(OH)CH₃, —COOCH₂CH(OH)CH₂OH, —COOCH₂CHXCH₂X, —CO(OCH₂CO)ₙOM, —CO[OCH(CH₃)CO]ₙOM —COOCH[CH(OH)]₄CH₂OH, —CO[C₆H₁₂O₆, sucrose], —CONR¹R², —COO(CH₂)₂NR¹R², —COO[CH(CH₃)CH₃]NR¹R ², —COOR³, or N-pyrrolidone; where X is H or RCOO—; M is H or a pharmaceutically acceptable counter ion; R¹ and R² are independently H, CH₃, C₂H₅, C₃H₇, C₂H₄OH, or C₃H₇OH; R³ is CH₃, C₂H₅, or C₃H₇; m is an integer of 2 to 6; and n is an integer of 1 to 4.

23. The transdermal composition of claim 21, wherein said co-enhancer is a member selected from the group consisting of fatty acids and their salts, fatty alcohols, branched aliphatic alcohols, fatty acid alkyl esters, fatty acid monoesters of sorbitol and glycerol, fatty acid esters with glycolic acid and lactylic acid and their salts, fatty acid amides, alkylpyrrolidones and mixtures thereof.

24. The transdermal composition of claim 21, wherein said co-enhancer is a member selected from the group consisting of oleic acid; lauric acid; oleyl alcohol; lauryl alcohol; 2-butyl-octanol; 2-hexyl decanol; 2-octyl-decanol; 2-hexyldodecanol; 2-octyl-dodecanol; 2-decyl-tetradecanol; 2-tetradecyl-octadecanol; methyl and ethyl laurate; sorbitan monooleate and monolaurate; glycerol monooleate and monolaurate; lauric, myristic, capric, stearic, and oleic diethanolamide; lauric, myristic, capric, stearic, and oleic monoethanolamide; lauric, myristic, capric, stearic, and oleic monoisopropanolamide; caproyl, lauroyl and stearoyl lactylic acid and their salts; caproyl, lauroyl and stearoyl glycolic acid and their salts; N-n-octyl and N-n-dodecyl pyrrolidone.

25. The transdermal composition of claim 21, wherein said co-enhancer is oleic acid; lauric acid; oleyl alcohol; lauryl alcohol; 2-butyl-octanol; sorbitan monooleate; glycerol monooleate; lauric, stearic, and oleic diethanolamide; lauric monoisopropanolamide; caproyl lactylic acid; N-n-octyl pyrrolidone, or a mixture thereof.

26. The transdermal composition of claim 1, wherein said drug is a member selected from the group consisting of: antibiotics, neoplastic agents, agents affecting the immune response, blood calcium regulators, peptide and protein hormones, agents useful in glucose regulation, antithrombotics and hemostatics, antihyperlipidemic agents, thyromimetic and antithyroid drugs, anti-ulcer agents, histamine H2-receptor agonists and antagonists, inhibitors of allergic response, local anesthetics, analgesics and analgesic combinations, antipsychotics, anti-anxiety agents, antidepressants agents, anorexigenics, bone-active agents, diagnostic agents, antidiarrheals, antimigraine agents, antimotion sickness agents, antinauseants, antiparkinsonism agents, antipruritics, antipyretics, antispasmodics, anticholinergics, sympathomimetics, xanthine derivatives, cardiovascular agents, central nervous system stimulants, decongestants, diagnostics, hormones, immunosuppressives, parasympatholytics, parasympathomimetics, sedatives, tranquilizers and mixtures thereof.

27. A transdermal composition comprising a pharmaceutically acceptable carrier, a drug, and a quaternary ammonium salt, wherein the quaternary ammonium salt constitutes an amount sufficient to enhance penetration of the drug with reduced skin irritation.

28. The transdermal composition of claim 27, wherein the quaternary ammonium salt is present in low concentration.

29. The transdermal composition of claim 28, wherein the low concentration represents no greater than 4.5% by weight of the carrier.

30. The transdermal composition of claim 28, wherein the low concentration represents no greater than 4.0% by weight of the carrier.

31. The transdermal composition of claim 28, wherein the low concentration represents no greater than 3.0% by weight of the carrier.

32. The transdermal composition of claim 28, wherein the low concentration represents no greater than 2.0% by weight of the carrier.

33. The transdermal composition of claim 28, wherein the low concentration represents no greater than 1.0% by weight of the carrier.

34. The transdermal composition of claim 28, wherein said quaternary ammonium salt is a compound having the formula: 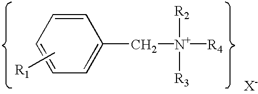
wherein R₁ is a member selected from the group consisting of H and C₁-C₁₂ straight or branched chain alkyl; R₂ and R₃ are independent members selected from the group consisting of CH₃, —CH₂OH and —CH₂ CH₂OH; R₄ is a member selected from the group consisting of:
(a) CH₃,
(b) C₂-C₂₂ straight or branched chain alkyl,
(c) C₂-C₂₂ straight or branched chain alkenyl,
(d) [CH₂CH₂O]ₙ—R₅ where n is an integer of 1-3 and R₅ is a member selected from the group consisting of H, C₁-C₁₂ straight or branched chain alkyl, C₂-C₂₂ straight or branched alkenyl; and 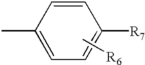
wherein R₆ is a member selected from the group consisting of H and —CH₃ and R₇ is a member selected from the group consisting of C₁-C₂₂ straight or branched chain alkyl and C₂-C₂₂ straight or branched chain alkenyl, and
(e)—(CH₂)ₘ NOCR₇ or —(CH₂)ₘ CONR₇ where m is an integer of 1-3; and
X is a pharmaceutically acceptable counter-ion.

35. The transdermal composition of claim 28, wherein said quaternary ammonium salt is benzalkonium chloride; benzalkonium saccharinate; behenalkonium chloride; cetalkonium chloride; erucalkonium chloride; lauralkonium chloride; myristalkonium chloride; myristalkonium saccharinate (Quaternium-3); stearalkonium chloride; olealkonium chloride; tallowalkonium chloride; dodecylbenzyltrimethylammonium chloride (Quaternium-28); dodecylbenzyl trimethyl ammonium 2-ethylhexanoate; ethylbenzyl alkyldimethylammonium cyclohexylsulfanamate (Quaternium-8); ethylbenzyl dimethyl dodecyl ammonium chloride (Quaternium-14); dodecylbenzyl dimethyl octadecyl ammonium chloride; dodecylbenzyl triethanol ammonium chloride (Quaternium-30); benzoxonium chloride; benzylbis(2-hydroxyethyl)(2-dodecyloxyethyl)ammonium bromide; benzylbis(2-hydroxyethyl)(2-dodecyloxyethyl)ammonium chloride; benzethonium chloride; methylbenzethonium chloride; N,N-(diethyl-N-[2-[4-( 1,1,3,3-tetramethylbutyl)phenoxy]ethyl] benzenemethanaminium chloride (phenoctide); dodecarbonium chloride; babassuamidopropalkonium chloride; wheatgermamidopropalkonium chloride, or a mixture thereof.

36. The transdermal composition of claim 28, wherein said quaternary ammonium salt is benzalkonium chloride, stearalkonium, behenalkonium chloride, olealkonium chloride, erucalkonium chloride, benzethonium chloride, methylbenzethonium chloride, phenoctide, wheatgermamidopropalkonium chloride, babassuamidopropalkonium chloride or a mixture thereof.

37. The transdermal composition of claim 28, wherein the quaternary ammonium salt is benzethonium chloride.

38. The transdermal composition of claim 28, wherein the counter-ion is selected from the group consisting of chloride, bromide, iodide , acetate, 2-ethylhexanoate, sulfate, phosphate, arylsulfonates, cyclohexylsulfamate, benzoate, saccharinate and a mixture thereof.

39. A method of reducing skin irritation of a transdermal composition comprising a pharmaceutically acceptable carrier,
comprising the step of incorporating a low concentration of a quaternary ammonium salt.

40. The method of claim 39, wherein the low concentration represents no greater than 4% by weight of the carrier.

41. The method of claim 39, wherein the low concentration represents no greater than 3% by weight of the carrier.

42. The method of claim 39, wherein the low concentration represents no greater than 2% by weight of the carrier.

43. The method of claim 39, wherein the low concentration represents no greater than 1% by weight of the carrier.

44. The method of claim 39, wherein the low concentration represents no greater than 0.8% by weight of the polymeric carrier.

45. The transdermal composition of claim 39, wherein said quaternary ammonium salt is a compound having the formula: 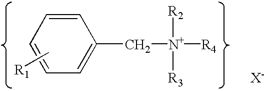
wherein R₁ is a member selected from the group consisting of H and C₁-C₁₂ straight or branched chain alkyl; R₂ and R₃ are independent members selected from the group consisting of CH₃, —CH₂OH and —CH₂ CH₂OH; R₄ is a member selected from the group consisting of:
(a) CH₃,
(b) C₂-C₂₂ straight or branched chain alkyl,
(c) C₂-C₂₂ straight or branched chain alkenyl,
(d) [CH₂CH₂O)]ₙ—R₅ where n is an integer of 1-3 and R₅ is a member selected from the group consisting of H, C₁-C₁₂ straight or branched chain alkyl, C₂-C₂₂ straight or branched alkenyl; and 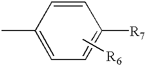
wherein R₆ is a member selected from the group consisting of H and —CH₃ and R₇ is a member selected from the group consisting of C₁-C₂₂ straight or branched chain alkyl and C₂-C₂₂ straight or branched chain alkenyl, and
(e)—(CH₂)ₘ NOCR₇ or —(CH₂)ₘ CONR₇ where m is an integer of 1-3; and
X is a pharmaceutically acceptable counter-ion.

46. The transdermal composition of claim 39, wherein said quaternary ammonium salt is benzalkonium chloride; benzalkonium saccharinate; behenalkonium chloride; cetalkonium chloride; erucalkonium chloride; lauralkonium chloride; myristalkonium chloride; myristalkonium saccharinate (Quaternium-3); stearalkonium chloride; olealkonium chloride; tallowalkonium chloride; dodecylbenzyltrimethylammonium chloride (Quaternium-28); dodecylbenzyl trimethyl ammonium 2-ethylhexanoate; ethylbenzyl alkyldimethylammonium cyclohexylsulfanamate (Quaternium-8); ethylbenzyl dimethyl dodecyl ammonium chloride (Quaternium-14); dodecylbenzyl dimethyl octadecyl ammonium chloride; dodecylbenzyl triethanol ammonium chloride (Quaternium-30); benzoxonium chloride; benzylbis(2-hydroxyethyl)(2-dodecyloxyethyl)ammonium bromide; benzylbis(2-hydroxyethyl)(2-dodecyloxyethyl)ammonium chloride; benzethonium chloride; methylbenzethonium chloride; N,N-(diethyl-N-[2-[4-(1,1,3,3-tetramethylbutyl)phenoxy]ethyl] benzenemethanaminium chloride (phenoctide); dodecarbonium chloride; babassuamidopropalkonium chloride; wheatgermamidopropalkonium chloride, or a mixture thereof.

47. The transdermal composition of claim 39, wherein said quaternary ammonium salt is benzalkonium chloride, stearalkonium, behenalkonium chloride, olealkonium chloride, erucalkonium chloride, benzethonium chloride, methylbenzethonium chloride, phenoctide, wheatgermamidopropalkonium chloride, babassuamidopropalkonium chloride or a mixture thereof.

48. The transdermal composition of claim 39, wherein the quaternary ammonium salt is benzethonium chloride.

49. The transdermal composition of claim 39, wherein the counter-ion is selected from the group consisting of chloride, bromide, iodide, acetate, 2-ethylhexanoate, sulfate, phosphate, arylsulfonates, cyclohexylsulfamate, benzoate, saccharinate and a mixture thereof.

50. A method of synergistically enhancing transdermal penetration of a drug in a transdermal composition comprising a carrier, a penetration enhancer, and a drug, comprising the step of incorporating a low concentration of a quaternary ammonium salt.

51. The method of claim 50, wherein the low concentration represents no greater than 4% by weight of the carrier.

52. The method of claim 50, wherein the low concentration represents no greater than 3% by weight of the carrier.

53. The method of claim 50, wherein the low concentration represents no greater than 2% by weight of the carrier.

54. The method of claim 50, wherein the low concentration represents no greater than 1% by weight of the carrier.

55. The method of claim 50, wherein the low concentration represents no greater than 0.8% by weight of the carrier.

56. The transdermal composition of claim 50, wherein said quaternary ammonium salt is a compound having the formula: 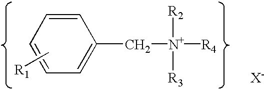
wherein R₁ is a member selected from the group consisting of H and C₁-C₁₂ straight or branched chain alkyl; R₂ and R₃ are independent members selected from the group consisting of CH₃, —CH₂OH and —CH₂ CH₂OH; R₄ is a member selected from the group consisting of:
(a) CH₃,
(b) C₂-C₂₂ straight or branched chain alkyl,
(c) C₂-C₂₂ straight or branched chain alkenyl,
(d) [CH₂CH₂O)]ₙ—R₅ where n is an integer of 1-3 and R₅ is a member selected from the group consisting of H, C₁-C₁₂ straight or branched chain alkyl, C₂ - C₂₂ straight or branched alkenyl; and 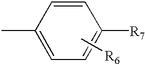
wherein R₆ is a member selected from the group consisting of H and —CH₃ and R₇ is a member selected from the group consisting of C₁-C₂₂ straight or branched chain alkyl and C₂-C₂₂ straight or branched chain alkenyl, and
(e)—(CH₂)ₘ NOCR₇ or —(CH₂)ₘ CONR₇ where m is an integer of 1-3; and
X is a pharmaceutically acceptable counter-ion.

57. The transdermal composition of claim 50, wherein said quaternary ammonium salt is benzalkonium chloride; benzalkonium saccharinate; behenalkonium chloride; cetalkonium chloride; erucalkonium chloride; lauralkonium chloride; myristalkonium chloride; myristalkonium saccharinate (Quaternium-3); stearalkonium chloride; olealkonium chloride; tallowalkonium chloride; dodecylbenzyltrimethylammonium chloride (Quaternium-28); dodecylbenzyl trimethyl ammonium 2-ethylhexanoate; ethylbenzyl alkyldimethylammonium cyclohexylsulfanamate (Quaternium-8); ethylbenzyl dimethyl dodecyl ammonium chloride (Quaternium-14); dodecylbenzyl dimethyl octadecyl ammonium chloride; dodecylbenzyl triethanol ammonium chloride (Quaternium-30); benzoxonium chloride; benzylbis(2-hydroxyethyl)(2-dodecyloxyethyl)ammonium bromide; benzylbis(2-hydroxyethyl)(2-dodecyloxyethyl)ammonium chloride; benzethonium chloride; methylbenzethonium chloride; N,N-(diethyl-N-[2-[4-(1,1,3,3-tetramethylbutyl)phenoxy]ethyl] benzenemethanaminium chloride (phenoctide); dodecarbonium chloride; babassuamidopropalkonium chloride; wheatgermamidopropalkonium chloride, or a mixture thereof.

58. The transdermal composition of claim 50, wherein said quaternary ammonium salt is benzalkonium chloride, stearalkonium, behenalkonium chloride, olealkonium chloride, erucalkonium chloride, benzethonium chloride, methylbenzethonium chloride, phenoctide, wheatgermamidopropalkonium chloride, babassuamidopropalkonium chloride or a mixture thereof.

59. The transdermal composition of claim 50, wherein the quaternary ammonium salt is benzethonium chloride.

60. The transdermal composition of claim 50, wherein the penetration enhancer comprises a compound represented by the formula:
R-Y
wherein R is a straight chain alkyl of about 7 to 17 carbon atoms, a non-terminal alkenyl of about 7 to 22 carbon atoms, or a branched-chain alkyl from about 12 to 22 carbons; and Y is —OH, —COOH, —OCOCH₃, —SOCH₃, —P(CH₃)₂O, —COO(C₂H₄O)ₘH, —(OC₂H₄)ₘOH, —COOCH₂CH(OH)CH₃, —COOCH₂CH(OH)CH₂OH, —COOCH₂CHXCH₂X, —CO(OCH₂CO)ₙOM, —CO[OCH(CH₂)CO]ₙOM —COOCH[CH(OH)]₄CH₂OH, —CO[C₆H₁₂O₆, sucrose], —CONR¹R², —COO(CH₂)₂NR¹R², —COO[CH(CH₃)CH₃]NR¹R², —COOR³, or N-pyrrolidone; where X is H or RCOO—; M is H or a pharmaceutically acceptable counter ion; R¹ and R² are independently H, CH₃, C₂H₅, C₃H₇, C₂H₄OH, or C₃H₇OH; R³ is CH₃, C₂H₅, or C₃H₇; m is an integer of 2 to 6; and n is an integer of 1 to 4.

61. The transdermal composition of claim 50, wherein said enhancer is a member selected from the group consisting of fatty acids and their salts, fatty alcohols, branched aliphatic alcohols, fatty acid alkyl esters, fatty acid monoesters of sorbitol and glycerol, fatty acid esters with glycolic acid and lactylic acid and their salts, fatty acid amides. alkylpyrrolidones and mixtures thereof.

62. The transdermal composition of claim 50, wherein said enhancer is a member selected from the group consisting of oleic acid; lauric acid; oleyl alcohol; lauryl alcohol; 2-butyl-octanol; 2-hexyl decanol; 2-octyl-decanol; 2-hexyldodecanol; 2-octyl-dodecanol; 2-decyl-tetradecanol; 2-tetradecyl-octadecanol; methyl and ethyl laurate; sorbitan monooleate and monolaurate; glycerol monooleate and monolaurate; lauric, myristic, capric, stearic, and oleic diethanolamide; lauric, myristic, capric, stearic, and oleic monoethanolamide; lauric, myristic, capric, stearic, and oleic monoisopropanolamide; caproyl, lauroyl and stearoyl lactylic acid and their salts; caproyl, lauroyl and stearoyl glycolic acid and their salts; N-n-octyl and N-n-dodecyl pyrrolidone.

63. The transdermal composition of claim 50, wherein said enhancer is oleic acid; lauric acid; oleyl alcohol; lauryl alcohol; 2-butyl-octanol; sorbitan monooleate; glycerol monooleate: lauric, stearic, and oleic diethanolamide; lauric monoisopropanolamide; caproyl lactylic acid ; N-n-octyl pyrrolidone, or a mixture thereof.

64. The transdermal composition of claim 50, wherein the counter-ion is selected from the group consisting of chloride, bromide, iodide, acetate, 2-ethylhexanoate, sulfate, phosphate, arylsulfonates, cyclohexylsulfamate, benzoate, saccharinate and a mixture thereof.

65. The transdermal composition of claim 1, wherein the drug is oxybutynin, buspirone, fentanyl, testosterone, progesterone, estradiol, propentofylline, or a mixture thereof, or a salt, isomer, or analog thereof.

66. The transdermal composition of claim 27, wherein the drug is oxybutynin, buspirone, fentanyl, testosterone, progesterone, estradiol, propentofylline, or a mixture thereof, or a salt, isomer, or analog thereof.

67. The transdermal composition of claim 39, wherein the drug is oxybutynin, buspirone, fentanyl, testosterone, progesterone, estradiol, propentofylline, or a mixture thereof, or a salt, isomer, or analog thereof.

68. The transdermal composition of claim 50, wherein the drug is oxybutynin, buspirone, fentanyl, testosterone, progesterone, estradiol, propentofylline, or a mixture thereof, or a salt, isomer, or analog thereof.

69. The method of claim 39, wherein the skin irritation manifests as erythema, papule, vesicle, or a combination thereof.

70. The method of claim 39, wherein the skin irritation is caused by microbial growth.

71. The method of claim 70, wherein the microbial comprises gram-positive bacteria.

72. The method of claim 50, wherein the penetration enhancement is from about 10-100% greater than would be expected of an additive effect from using the quaternary ammonium salt and a penetration enhancer.

73. The method of claim 50, wherein the penetration enhancement is from about 20-100% greater than would be expected of an additive effect from using the quaternary ammonium salt and a penetration enhancer.

74. The method of claim 50, wherein the penetration enhancement is from about 10-50% greater than would be expected of an additive effect from using the quaternary ammonium salt and a penetration enhancer.

75. A method of enhancing transdermal delivery of a drug and reducing skin irritation associated with the transdermal delivery comprising the step of:
applying a transdermal drug delivery system as recited in claim 1 to a selected skin surface.

76. A transdermal composition for reducing skin irritation, comprising a low concentration of a quaternary ammonium salt, wherein the composition results in no greater than mild skin irritation when applied to the skin.
